(19) United States
(12) Patent Application Publication
(10) Pub. No.: US 2015/0152458 A1
(43) Pub. Date: Jun. 4, 2015

(54) LOW TEMPERATURE METHOD FOR MAKING HIGH GLUCOSE SYRUP

(71) Applicant: Danisco US Inc., Palo Alto, CA (US)

(72) Inventors: Bart Christian Koops, Pijnacker (NL); Floor K. Kooy, Leiden (NL); Sung Ho Lee, North Liberty, IA (US); Jayarama K. Shetty, Pleasanton, CA (US); Bruce A. Strohm, Beloit, WI (US); Arjen Hendrik van Tuijl, Zoetermeer (NL)

(73) Assignee: DANISCO US INC., Palo Alto, CA (US)

(21) Appl. No.: 14/479,653

(22) PCT Filed: Mar. 26, 2013
(86) PCT No.: PCT/US2013/033853

§ 371 Date: Sep. 8, 2014

Related U.S. Application Data
(60) Provisional application No. 61/616,980, filed on Mar. 28, 2012.

(51) Int. Cl. C12P 19/20

(52) U.S. Cl. CPC C12P 19/20

(57) ABSTRACT

The present teachings provide a method for making a high glucose syrup at a low temperature. In some embodiments, the syrup contains reduced reversion reaction products. The method comprises contacting a starch substrate at a temperature below the starch gelatinization temperature with an enzyme blend comprising a high dose of alpha-amylase and a low dose of glucoamylase. In some embodiments, a blend of two glucoamylases is employed. In some embodiments, a debranching enzyme such as a pullulanase is employed. In some embodiments, the enzymes are staged by adding at different times, or at different temperatures. The present teachings provide for high glucose syrups with fewer reversion products, and allow for higher starch solubilization.

### FIELD OF THE INVENTION

This disclosure is directed towards improved methods and compositions for making high glucose-containing syrups from refined starch substrates.

### BACKGROUND OF THE INVENTION

Extensive process optimization has been done on the large scale production of high glucose syrup over the last many years to improve quality and process economics (T. W. Martin and Brumm, P. J 1992 “Commercial enzymes for starch hydrolysis products” in Starch Hydrolysis Products: Worldwide Technology, production and applications 45-77 New York, VCH Publishers, Inc.; Luenser, S. J, 1983 Microbial enzymes for Industrial sweetener production, Dev. in Ind. Microbiol. 24. 79-96).

Further industrial processes have been adopted by the starch sweetener industry for the enzyme liquefaction process (U.S. Pat. No. 5,322,778). Some of these processes include a low temperature process (105-110° C. for 5-8 min) with lower steam requirements and a high temperature process (148° C.+/−5 C for 8-10 sec), which improves gelatinization of the starch granules resulting in improved filtration characteristics and quality of the liquefied starch substrate (Shetty, et al., (1988) Cereal Foods World 33:929-934). Further improvement in the liquefaction process has been demonstrated by multiple additions of thermostable alpha amylases, pre- and post jet cooking steps which significantly resulted in the improvements with respect to yield loss, processing costs, energy consumption, pH adjustments, temperature thresholds, calcium requirement and levels of retrograded starch.

In the late 1950s, glucoamylases derived from Aspergillus niger were commercialized and these enzymes significantly improved the conversion of solubilized/liquefied starch substrate to glucose at a pH between pH 4.0 to 5.0 and a temperature of 20-65° C. Commercial glucose syrup is generally produced in high yields by a two-step enzymatic hydrolysis of starch under two different pH conditions because of the differences in the pH stability of the liquefaction and saccharification enzymes system. In this “conventional process”, the pH of the hydrolysate is decreased to pH 4.0-4.6 to fit in to the optimum pH for glucoamylase from fungal source, i.e. Aspergillus niger or Trichoderma reesei (e.g. OPTIDEX® L-400 ,G-ZYME® 480 Ethanol, GC 147 from Danisco-Genencor) to convert low DE substrate to glucose. Glucoamylase is an exo-acting enzyme, releasing glucosyl residues in step-wise from non-reducing ends of starch substrates. The enzyme has a higher affinity for high molecular weight starch resulting in a rapid starch hydrolysis rate that decreases with decreasing molecular weight of oligosaccharides. In addition, the amylopectin that represents over 80% of starch contains branch points connecting linear amylose through alpha 1-6 glycosidic linkages. Commercially available glucoamylases are very fast in hydrolyzing alpha 1-4 glycosidic linkages in high molecular weight starch substrate, and the rate decreases (Km increases) with decreasing molecular weight of the oligosaccharides. This requires a relatively high dose of glucoamylases and/or longer saccharification time for completing the hydrolysis. It is also known that the rate of hydrolysis of alpha 1-6 (branch) linkages in amylopectin is much slower compared to the rate of hydrolysis of alpha 1-4 glycosidic linkages by glucoamylase. Even though starch contains only 3.5 to 4.0% of alpha 1-6 linkages, the resistance on the hydrolysis of liquefied starch by glucoamylase is very significant. The introduction of pullulanase (debranching enzyme) in the mid-1980's, an enzyme which is very specific at catalyzing the hydrolysis of the branch point in amylopectin, resulted in a significant improvement in the efficiency of glucose production. For example, a significant improvement in the glucose production process was accomplished by the introduction of an acid-stable thermostable debranching enzyme during saccharification (For example, OPTIMAX® L-1000 from Danisco-Genencor and Promozyme™® and Dextrozyme® blends from Novozymes Inc.)

Another problem associated with glucoamylase catalysis of soluble starch substrate is that the majority of saccharification time (more than 70%) is spent increasing the glucose yield from 85% to 96%. This is mainly due to difficulty of glucoamylase in hydrolyzing the low molecular weight soluble oligosaccharides such as DP2, DP3, and DP4 etc., thus requiring relatively high dose of glucoamylase or longer saccharification time to maximize glucose production.

The industrial needs for increased productivity, improved quality and reduced energy cost for evaporation still exist. For example, liquefaction is generally carried out using a starch slurry at a dry solid content greater than 35%, however the liquefied starch substrate necessitates further diluting to a lower dissolved solids (e.g. 32% for saccharification for obtaining a glucose yield greater than 95.5%). This material is concentrated by evaporation, refined and processed to final products of high glucose syrup, crystalline dextrose or high fructose syrup (Habeda, R. E; In Kirk-Othmer Encyclopedia of Chemical Technology” Vol 22, Third Edition. John Wiley &Sons, Inc. New York 1983, pp 499-522). Additionally, industrial needs for improvements with respect to yield loss, processing costs, lowering the energy consumption, pH adjustments, and reducing the high risk of blue sac resulting from retrograded starch during the saccharification still exist. Direct conversion of the uncooked starch/granular starch using granular starch hydrolyzing enzyme composition has been suggested. For example U.S. Pat. No. 4,618,579 (Dwiggins et.al; 1986;) and U.S. Pat. No. 7,303,899 (Baldwin; et.al 2007) disclosed a process for producing high glucose syrup using granular starch without jet cooking with an composition containing Humicola grisea glucoamylase and Bacillus stearothermophilus liquefying alpha-amylase.

Glucose manufacturers have been constantly looking for ways to conduct saccharification at higher dissolved solids to reduce the energy cost of evaporation, and to improve the plant production capacity. Saccharification at higher dissolved solids (e.g. 32% DS and greater) is known to promote the reversion reaction catalyzed by glucoamylase, and result in the accumulation of branched saccharides that are not readily hydrolyzed by glucoamylase. This results in lower glucose. The reversion reaction catalyzed by glucoamylase results in high DP2 sugars levels containing isomaltose, kojibiose, and nigerose. Three major factors impact these DP2 levels: dry solids, glucose concentration, and glucoamylase dose. The formation of these reversion reaction products not only results in lower product yield but also affect the quality of the final product.

Inefficient liquefaction of starch substrate generally results in a higher level of retrograded starch in the starch substrate for saccharification. This retrograded starch is resistant to hydrolysis by conventional saccharification enzymes like glucoamylase or glucoamylase blends containing pullulanases, resulting in iodine positive glucose syrup (generally called “Blue Sac”).The iodine positive glucose syrup is not widely accepted in commerce because of problems associated with its processing and functionality.

In summary, unmet commercial needs remain for improving the conventional process for converting starch substrate into high glucose which include: elimination of sulphuric acid addition for pH adjustment, thereby providing a single step for converting granular starch to high glucose syrup containing reduced reversion reaction products; elimination of the step for inactivating the residual liquefying alpha-amylase activity prior to saccharification thereby producing a final glucose syrup containing low levels of DP3; and, hydrolysis of starch substrate at high dissolved solids thereby allowing for a reduced level of glucoamylase-catalyzed reversion reaction products.

All patents, patent applications, publications, documents, nucleotide and protein sequence database accession numbers, the sequences to which they refer, and articles cited herein are all incorporated herein by reference in their entireties.

### BRIEF SUMMARY OF THE INVENTION

The present teachings provide a method of making a glucose syrup from refined granular starch slurry comprising; contacting the refined granular starch slurry at a temperature at or below the initial starch gelatinization temperature with a dose of at least 8 AAU/gds of an alpha-amylase, and, a dose of 0.05 GAU/gds to no more than 0.3 GAU/gds of glucoamylase, and, making a glucose syrup.

Additional methods, as well as compositions, are also provided.

### BRIEF DESCRIPTION OF THE DRAWING

FIG. 1 depicts the lower DP2 levels produced by the methods of the present teachings (diamonds) compared to conventional methods (squares).

### DETAILED DESCRIPTION

The invention provides, inter alia, a method of making a glucose syrup from refined granular starch slurry comprising: contacting the refined granular starch slurry at temperature below the starch gelatinization temperature with a dose of at least 8 AAU/gds of an alpha-amylase, and, a dose of 0.05 GAU/gds to no more than 0.3 GAU/gds of glucoamylase, and, making a glucose syrup.

In some embodiments, the glucose syrup comprises a DP1 of at least 90%. In some embodiments, at least 80% of the refined granular starch is solubilized.

In some embodiments, the glucose syrup comprises a DP2 of less than 3%.

In some embodiments, the refined granular starch slurry comprises an initial DS of 31%-44% or 33-37%.

In some embodiments, the glucoamylase comprises a mixture of glucoamylases, the mixture comprising a fast hydrolyzing glucoamylase and a low reversion glucoamylase.

In some embodiments, the fast hydrolyzing glucoamylase is Humicola glucoamylase and molecules 97% identical thereto, and the low reversion glucoamylase is A. Niger glucoamylase and molecules 97% identical thereto.

In some embodiments, the method of the present teachings further comprise treating with a pullulanase.

In some embodiments, the pullulanase, if present, is at a dose of 0.2 ASPU/gds.

In some embodiments, the pullulanase dose is 0.15-0.25 ASPU/gds. In some embodiments, the pullulanase dose is 0.1-0.3 ASPU/gds.

In some embodiments, the pullulanase, if present, is Bacillus deramificans pullulanase and molecules 97% identical thereto.

In some embodiments, the present teachings comprise enzymes staging. For example, in some embodiments, a first dose of alpha-amylase is followed by a second dose of alpha-amylase, wherein the second dose occurs between 18 and 48 hours after the first dose. In some embodiments, a first dose of glucoamylase is followed by a second dose of glucoamylase, wherein the second dose occurs between 18 and 48 hours after the first dose.

In some embodiments, the present teachings comprise temperature staging. For example, in some embodiments, a first dose of alpha-amylase is applied at a first temperature, and the first temperature is elevated by 2° C.-8° C. after between 18 hours and 34 hours to a second temperature. In some embodiments, a first dose of glucoamylase is applied at a first temperature, and wherein the first temperature is elevated by 2° C.-8° C. after between 18 hours and 34 hours to a second temperature.

In some embodiments, the alpha-amylase is selected from the group consisting of B. stearothermophilus, B. amyloliquefaciens and B. licheniformis, and molecules 97% identical thereto. In some embodiments, the alpha-amylase is B. stearothermophilus wild-type, or molecules 97%, 98%, or 99% identical thereto.

In some embodiments, the glucose syrup is made in less than 60 hours.

In some embodiments, the present teachings provide compositions. For example, in some embodiments, the present teachings provide a composition comprising at least 8 AAU/gds of an alpha-amylase and 0.05 GAU/gds to no more than 0.3 GAU/gds of glucoamylase. In some embodiments, the composition further comprises refined granular starch. In some embodiments, the composition comprises a pullulanase. In some embodiments, the 0.05 GAU/gds to no more than 0.3 GAU/gds of glucoamylase comprises a first glucoamylase and a second glucoamylase.

In some embodiments, the dose of alpha-amylase is at least 9 AAU/gds. In some embodiments, the dose of alpha-amylase is at least 10, 11, 12, 13, 14, 15, 16, 17, 18, 19, 20, 21, 22, 23, 24, 25, 30, 35, 40, 45, 50, 60, 70, 80, 90, or 100 AAU/gds.

In some embodiments, the dose of glucoamylase is below 0.5 GAU/gds. In some embodiments, the dose of glucoamylase is below 0.45, 0.4, 0.35, 0.3, 0.25, 2, 0.15, 0.1, 0.05, 0.025, or 0.01 GAU/gds.

In some embodiments, the glucose syrup comprises a DP1 of at least 90%. In some embodiments, the glucose syrup comprises a DP1 of at least 91%, 92%, 93%, 94%, or 95%.

In some embodiments, at least 80% of the refined granular starch is solubilized.

In some embodiments, at least 81%, 82%, 83%, 84%, 85%, 86%, 87%, 88%, 89%, or 90% is solubilized.

In some embodiments, the glucose syrup comprises a DP2 of less than 3%. In some embodiments, the glucose syrup comprises a DP2 of less than 2.9, 2.8, 2.7, 2.6, 2.5, 2.4, 2.3, 2.2, 2.1, 2.0, 1.9, 1.8, 1.7, 1.6, or 1.5%.

In some embodiments, the initial DS of the refined granular starch slurry is 31%-44%.

In some embodiments, the initial DS of the refined granular starch slurry is 33%-37%.

In some embodiments, the initial DS of the refined granular starch slurry is 34%-36%.

In some embodiments, the glucoamylase comprises a mixture of Humicola glucoamylase and A. Niger glucoamylase.

In some embodiments, the method further comprises treating with a pullulanase.

In some embodiments, the pullulanase is Bacillus deramificans pullulanase.

In some embodiments, the method further comprises treating with a first dose of alpha-amylase followed by a second dose of alpha-amylase, wherein the second dose occurs between 18 and 48 hours. In some embodiments, the second occurs after 20, 22, 24, 26, 28, 30, 32, 34, 36, 38, 40, 42, 44, 46, or 48 hours.

In some embodiments, the method further comprises treating with a first dose of glucoamylase followed by a second dose of glucoamylase, wherein the second dose occurs between 18 and 48 hours. In some embodiments, the second occurs after 20, 22, 24, 26, 28, 30, 32, 34, 36, 38, 40, 42, 44, 46, or 48 hours.

In some embodiments, heat inactivation of the alpha-amylase is not required.

In some embodiments, the alpha-amylase is selected from the group consisting of B. stearothermophilus, B. amyloliquefaciens and B. licheniformis. In some embodiments, the alpha-amylase is SPEZYME® XTRA.

In some embodiments, the glucose syrup is made in less than 80 hours. In some embodiments, the glucose syrup is made in less than 75, 70, 65, 60, 55, 50, 45, 40, 35, or 30 hours.

In some embodiments of the present teachings, the reaction can be conducted at a temperature higher than the initial gelatinization temperature of a given starch. For example, in some embodiments the reaction is at 1, 2, 3, 3, 4, 5, 6, 7, 8, 9, 10, 11, 12, 13, 14, 15, 16, 17, 18, 19, or 20 degrees higher than the initial gelatinization temperature. In some embodiments, the reaction can be performed 1-5, 1-10, 5-10, 1-15, 5-15, or 1-20 degrees higher than the initial gelatinization temperature.

In some embodiments of the present teachings, the reaction can be conducted at a temperature lower than the initial gelatinization temperature of a given starch. For example, in some embodiments the reaction is at 1, 2, 3, 3, 4, 5, 6, 7, 8, 9, 10, 11, 12, 13, 14, 15, 16, 17, 18, 19, or 20 degrees lower than the initial gelatinization temperature. In some embodiments, the reaction can be performed 1-5, 1-10, 5-10, 1-15, 5-15, or 1-20 degrees lower than the initial gelatinization temperature.

In some embodiments, the refined starch of the present methods or compositions is from corn, wheat, barley, rye, triticale, sorghum, rice, oat, beans, banana, potato, sweet potato or tapioca. In some embodiments, the refined starch of the present methods or compositions is from corn.

In some embodiments, following treatment with enzymes according to the present teachings, any residual undissolved starch can be subsequently used as a fermentation feedstock. For example the undissolved starch can be subjected to conventional liquefaction to form a liquefact that microbes can ferment to form various biochemicals, including for example ethanol, lactic acid, succinic acid, citric acid, monosodium glutamate, 1-3 propanediol, and the like. In some embodiments, the undissolved starch can be re-treated with the same enzymes used in a low temperature first treatment to create a syrup and/or fermentable substrate.

In some embodiments, the present teachings provide a method of making a glucose syrup from refined granular starch slurry from corn comprising; contacting the refined granular starch slurry of 33-37% initial DS at a temperature at or below the initial starch gelatinization temperature with a dose of at least 8 AAU/gds of a Bacillus stearothermophilus alpha-amylase, a dose of 0.05 GAU/gds to no more than 0.3 GAU/gds of glucoamylase, wherein the glucoamylase comprises a first glucoamylase from Humicola grisea and a second glucoamylase from A. Niger, and, a dose of 0.15-0.25 ASPU/gds of Bacillus deramificans pullulanase; and, making a glucose syrup, wherein the glucose syrup comprises a DP2 of less than 3%.

In some embodiments, the present teachings provide a composition comprising refined granular starch slurry from corn, at least 8AAU/gds of a Bacillus stearothermophilus alpha-amylase, 0.05 GAU/gds to no more than 0.3 GAU/gds of glucoamylase, wherein the glucoamylase comprises equivalent GAU/gds of a first glucoamylase from Humicola grisea thermoida and a second glucoamylase from A. Niger, and 0.15-0.25 ASPU/gds of Bacillus deramificans pullulanase

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., “Dictionary of Microbiology and Molecular Biology” 2nd ed., J. Wiley & Sons (New York, N.Y. 1994), and Baltz et al., “Manual of Industrial Microbiology and Biotechnology” 3^{rd} ed., (Washington, D.C.: ASM Press, 2010), provide one skilled in the art with a general guide to many of the terms used in the present application.
1) Carbohydrate composition by High Pressure Liquid Chromatographic (HPLC) method. The composition of the reaction products of oligosaccharides was measured by high pressure liquid chromatography (HPLC) using either method (a) or (b) as described below. Similar results are obtained for a given sample using either method (a) or method (b). Sample preparation of the slurry sample involves centrifugation for 5 minutes at 13000 rpm, dilution of the syrup to a 3% solution, and denaturation of enzymes by boiling for 10 minutes. Samples were cooled down and then filtered using a 0.22 μm disc filter (Titan Syringe Filter PTFE, 0.45 μm 30 mm) prior to HPLC analysis. In both methods, the HPLC column used separates saccharides by molecular weight. For example a designation of DP1 is a monosaccharide, such as glucose; a designation of DP2 is a disaccharide, such as maltose; a designation of DP3 is a trisaccharide, such as maltotriose and the designation “DP3⁺” is an oligosaccharide having a degree of polymerization (DP) of 4 or greater. Area percentages of the different saccharides (DP3+, DP3, DP2, DP1) are calculated by dividing the area of each individual saccharide by the total area of all saccharides.
   a. An HPLC system (Beckman System Gold 32 Karat Fullerton, Calif., USA) equipped with a HPLC column (Rezex RCM-Monosaccharides), maintained at 80° C. fitted with a refractive index (RI) detector was used. De-ionized water was used as the mobile phase at a flow rate of 0.6 ml per minute. Twenty microliter of 3.0% solution was injected on to the column.
   b. An HPLC system (Prominence modular HPLC from Shimadzu Corporations; Kyoto, Japan) equipped with a HPLC column (Rezex RHM-monosaccharide H+ (8%) phase from Phenomenex, Inc.; Torrance, Calif., USA) maintained at 85° C. was used. Ultrapure, demineralized water (MilliQ) was used as the mobile phase at a flow rate of 0.6 ml per minute. For each sample, 5 microliter of 10% syrup solution was injected on to the column.
2) One AAU of bacterial alpha-amylase activity is the amount of enzyme required to hydrolyze 10 mg of starch per min from 5% dry solids soluble Lintner starch solution containing 31.2 mM calcium chloride, at 60° C. and 6.0 pH buffered with 30 mM sodium acetate.
3) One Glucoamylase Unit (GAU) is the amount of enzyme that liberates one gram of reducing sugars calculated as glucose from a 2.5% dry solids soluble Lintner starch substrate per hour at 60° C. and 4.3 pH buffered with 20 mM sodium acetate.
4) Percent solubilization of granular starch. Solubilization testing is done by sampling from the agitated slurry into two 2.5 ml micro-centrifuge tubes. One tube is spun for ˜4 minutes at 13,000 rpm and the refractive index of the supernatant is determined at 30° C. (RIₛᵤₚ). The total dry substance is determined by adding 1 drop of SPEZYME® FRED from a micro disposable-pipette to the second tube, then boiling 10 minutes. The tube is cooled and dry substance determined at 30° C. (RIₜₒₜ). The dry substance of the supernatant and the whole sample (total) are determined using appropriate DE tables. Table for converting RIₛᵤₚ to DS is the 95 DE, Table I from the Critical Data Tables of the Corn Refiners Association, Inc. To convert RIₜₒₜ to DS, more than one table can be used and an interpolation between the 32 DE and 95 DE tables employed. First an estimation of the solubilization is made by dividing the DS from the supernatant by the starting DS*1.1. The starting DS is the target dry substance starch slurry in the preparation and typically confirmed by Baume/DS tables or by dry substance determined on the original slurry by loss on drying (infrared balance). This estimated solubilization is used for the interpolation between the DS obtained via the 95 DE and 32 DE table. Solubilization is defined as the dry substance of the supernatant divided by the total dry substance times 100. This value is then corrected to compensate for the impact of remaining granular starch. This correction compensates for the water uptake by partial swelling and hydrolysis of starch granules remaining in the spin tube from the DS determination for supernatant.
5) One Acid Stable Pullulanase Unit (ASPU) is the amount of enzyme which liberates one equivalent reducing potential as glucose per minute from pullulan at pH 4.5 and a temperature of 60° C.
6) As used herein, “Liquefon unit” (LU) refers to the digestion time required to produce a color change with iodine solution, indicating a definite stage of dextrinization of starch substrate under standard assay conditions. In brief, the substrate can be soluble Lintner starch 5 g/L in phosphate buffer, pH 6.2 (42.5 g/liter potassium dihydrogen phosphate, 3.16 g/liter sodium hydroxide). The sample is added in 25 mM calcium chloride and activity is measured as the time taken to give a negative iodine test upon incubation at 30° C. Activity is recorded in liquefons per gram or mL (LU) calculated according to the formula:

$$LU \text{/} mL or LU \text{/} g = \frac{570}{V \times t} \times D$$

Where LU=liquefon unit; V=volume of sample (5 mL); t=dextrinization time (minutes); D=dilution factor=dilution volume/mL or g of added enzyme.
7) One “Modified Wohlgemuth unit” (MWU) refers to the amount of enzyme, e.g., Fuelzyme®-LF, which is able to hydrolyze 1 mg of soluble starch to specific dextrins under standard reaction conditions in 30 minutes.

### Definitions

As used herein the term “starch” refers to any material comprised of the complex polysaccharide carbohydrates of plants, comprised of amylose and/or amylopectin with the formula (C₆H₁₀O₅)ₓ, wherein X can be any number. In particular, the term refers to any plant-based material including but not limited to grains, grasses, tubers and roots and more corn, wheat, barley, rye, triticale, sorghum, rice, oat, beans, banana, potato, sweet potato or tapioca. After processing to purify the complex polysaccharide carbohydrates from the other plant molecules, it is called “refined starch”.

The term “granular starch” refers to uncooked (raw) starch, which has not been subject to gelatinization.

The term “starch gelatinization” means solubilization of a starch molecule to form a viscous suspension.

The term “Initial gelatinization temperature” refers to the lowest temperature at which gelatinization of a starch substrate begins. The exact temperature can be readily determined by the skilled artisan, and depends upon the specific starch substrate and further may depend on the particular variety of plant species from which the starch is obtained and the growth conditions. According to the present teachings, the initial gelatinization temperature of a given starch is the temperature at which birefringence is lost in 5% of the starch granules using the method described by Gorinstein. S. and Lii. Cl., Starch/Stark, Vol 44 (12) pp. 461-466 (1992). The initial starch gelatinization temperature ranges for a number of granular starches which may be used in accordance with the processes herein include barley (52-59° C.), wheat (58-64° C.), rye (57-70° C.), corn (62-72° C.), high amylose corn (67-80° C.), rice (68-77° C.), sorghum (68-77° C.), potato (58-68° C.), tapioca (59-69° C.) and sweet potato (58-72° C.) (Swinkels, pg. 32-38 in STARCH CONVERSION TECHNOLOGY, Eds Van Beynum et al., (1985) Marcel Dekker Inc. New York and The Alcohol Textbook 3.sup.rd ED. A Reference for the Beverage, Fuel and Industrial Alcohol Industries, Eds Jacques et al., (1999) Nottingham University Press, UK). Gelatinization involves melting of crystalline areas, hydration of molecules and irreversible swelling of granules. The gelatinization temperature occurs in a range for a given grain because crystalline regions vary in size and/or degree of molecular order or crystalline perfection. STARCH HYDROLYSIS PRODUCTS Worldwide Technology, Production, and Applications (eds/Shenck and Hebeda, VCH Publishers, Inc, New York, 1992) at p. 26.

The term “DE” or “dextrose equivalent” is an industry standard for measuring the concentration of total reducing sugars, calculated as D-glucose on a dry weight basis. Unhydrolyzed granular starch has a DE that is essentially 0 and D-glucose has a DE of 100.

The term “glucose syrup” refers to an aqueous composition containing glucose solids. In one embodiment, glucose syrup will include at least 90% D-glucose and in another embodiment glucose syrup will include at least 95% D-glucose. In some embodiments the terms glucose and glucose syrup are used interchangeably.

The term “total sugar content” refers to the total sugar content present in a starch composition.

The term “dry solids content (DS)” refers to the total solids (dissolved and undissolved) of a slurry (in %) on a dry weight basis. At the onset, “initial DS” refers to the dry solids in the slurry at time zero. As the hydrolysis reaction proceeds, the portion of DS that are dissolved can be referred to as “Syrup DS” as well as “Supernatant DS”.

The term “slurry” is an aqueous mixture containing unsolubilized starch granules.

The term “dry substance starch” or “dry solids starch” refers to the total starch solids of a slurry (in %) on a dry weight basis, subtracting out contributions from other significant macromolecules (e.g. protein).

The term “alpha-amylase” (E.C. class 3.2.1.1) refers to enzymes that catalyze the hydrolysis of alpha 1,4-glycosidic linkages. These enzymes have also been described as those effecting the exo or endohydrolysis of 1,4-α-D-glycosidic linkages in polysaccharides containing 1,4-α-linked D-glucose units. Another term used to describe these enzymes is glycogenase. Exemplary enzymes include alpha-1,4-glucan 4-glucanohydrase glucanohydrolase. In some of the embodiments encompassed by the invention, the alpha-amylase is a microbial enzyme having an E.C. number, E.C. 3.2.1.1-3 and in particular E.C. 3.2.1.1. In some embodiments, the alpha-amylase is a thermostable bacterial alpha-amylase. Suitable alpha-amylases may be naturally occurring as well as recombinant and mutant alpha-amylases. In particularly preferred embodiments, the alpha-amylase is derived from a Bacillus species. Preferred Bacillus species include B. subtilis, B. stearothermophilus, B. lentus, B. licheniformis, B. coagulans, and B. amyloliquefaciens (U.S. Pat. No. 5,763,385; U.S. Pat. No. 5,824,532; U.S. Pat. No. 5,958,739; U.S. Pat. No. 6,008,026 and U.S. Pat. No. 6,361,809). Particularly preferred alpha-amylases are derived from Bacillus strains B. stearothermophilus, B. amyloliquefaciens and B. licheniformis. Also reference is made to strains having ATCC 39709; ATCC 11945; ATCC 6598; ATCC 6634; ATCC 8480; ATCC 9945A and NCIB 8059. Commercially available alpha-amylases contemplated for use in the methods of the invention include; SPEZYME®AA; SPEZYME® FRED; G-ZYME® G997 (Genencor International Inc.) and TERMAMYL® 120-L, TERMAMYL® LC, TERMAMYL® SC and Liquozyme SUPRA (Novozymes).

The term “glucoamylase” refers to the amyloglucosidase class of enzymes (EC.3.2.1.3, glucoamylase, alpha-1,4-D-glucan glucohydrolase). These are exo-acting enzymes, which release glucosyl residues from the non-reducing ends of amylose and amylopectin molecules. The enzymes also hydrolyze alpha-1,6 and alpha-1,3 linkages although at much slower rates than alpha-1,4 linkages. Glucoamylases (E.C. 3.2.1.3) are enzymes that remove successive glucose units from the non-reducing ends of starch. The enzyme can hydrolyze both linear and branched linkages of starch, amylose and amylopectin. While glucoamylase may be derived from bacteria, plants and fungi, preferred glucoamylases encompassed by the present are derived from fungal strains. Glucoamylases secreted from fungi of the genera Aspergillus, Rhizopus, Humicola and Mucor have been derived from fungal strains, including Aspergillus niger, Aspergillus awamori, Rhizopus niveus, Rhizopus oryzae, Mucor miehe, Humicola grisea, Aspergillus shirousami and Humicola (Thermomyces) laniginosa (See, Boel et al. (1984) EMBO J. 3:1097-1102; WO 92/00381; WO 00/04136; Chen et al., (1996) Prot. Eng. 9:499-505; Taylor et al., (1978) Carbohydrate Res. 61:301-308 and Jensen et al., (1988) Can. J. Microbiol. 34:218-223). Enzymes having glucoamylase activity used commercially are produced for example, from Aspergillus niger (trade name OPTIDEX® L-400 and G-ZYME® G990 4X from Genencor International Inc., presented herein as A-GA and An-GA) or Rhizopus species (trade name CU CONC®^{?} from Shin Nihon Chemicals, Japan and trade name GLUCZYME® from Amano Pharmaceuticals, Japan). Recombinantly expressed Humicola GA (H-GA) is from a Trichoderma host as described in U.S. Pat. No. 7,303,899 was used. In other embodiments the Trichoderma host expresses a heterologous polynucleotide which encodes a Humicola grisea strain, particularly a strain of Humicola grisea var. thermoidea. In some embodiments, a CS4 variant of Trichoderma can be employed (for example as taught in U.S. Pat. No. 8,058,033), as well as other variants including Brew 1 and Brew 11 (for example as taught in WO2011/020852 and WO2012/001139).

As used herein, the term “Pullulanase” (also called Debranching enzyme (E.C. 3.2.1.41, pullulan 6-glucanohydrolase)) is an enzyme capable of hydrolyzing alpha 1-6 glycosidic linkages in an amylopectin molecule. These enzymes are generally secreted by a Bacillus species; for example, Bacillus deramificans (U.S. Pat. No. 5,817,498; 1998), Bacillus acidopullulyticus (European Patent # 0 063 909 and Bacillus naganoensis (U.S. Pat. No. 5,055,403). Enzymes having pullulanase activity used commercially are produced from, for example, Bacillus species (trade name OPTIMAX® L-1000 from Danisco-Genencor and Promozyme® from Novozymes) or from Bacillus megaterium amylase/transferase (BMA). Bacillus megaterium amylase has the ability to convert the branched saccharides to a form that is easily hydrolyzed by glucoamylase (Habeda R. E, Styrlund C. R and Teague. M; 1988 Starch/Starke, 40,33-36). The enzyme exhibits maximum activity at pH 5.5 and temperature at 75 C (David, M. H, Gunther H and Vilvoorde, H. R; 1987, Starch/Starke, 39 436-440). The enzyme has been cloned, expressed in a genetically engineered Bacillus subtilis and produced on a commercial scale (Brumm, P. J, Habeda R. E, and Teague W. M, 1991 Starch/Starke, 43 315-329). The enzyme is sold under a trade name Megadex for enhancing the glucose yield during the saccharification of enzyme liquefied starch by Aspergillus niger glucoamylase. In other embodiments the Trichoderma host expresses a heterologous polynucleotide which encodes a Humicola grisea strain, particularly a strain of Humicola grisea var. thermoidea.

The term “hydrolysis of starch” refers to the cleavage of glycosidic bonds with the addition of water molecules.

The term “degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP3⁺ (>DP3) denotes polymers with a degree of polymerization of greater than 3.

The term “contacting” refers to the placing of the respective enzymes in sufficiently close proximity to the respective substrate to enable the enzymes to convert the substrate to the end product. Those skilled in the art will recognize that mixing solutions of the enzyme with the respective substrates can effect contacting.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### EXAMPLES

### Example 1

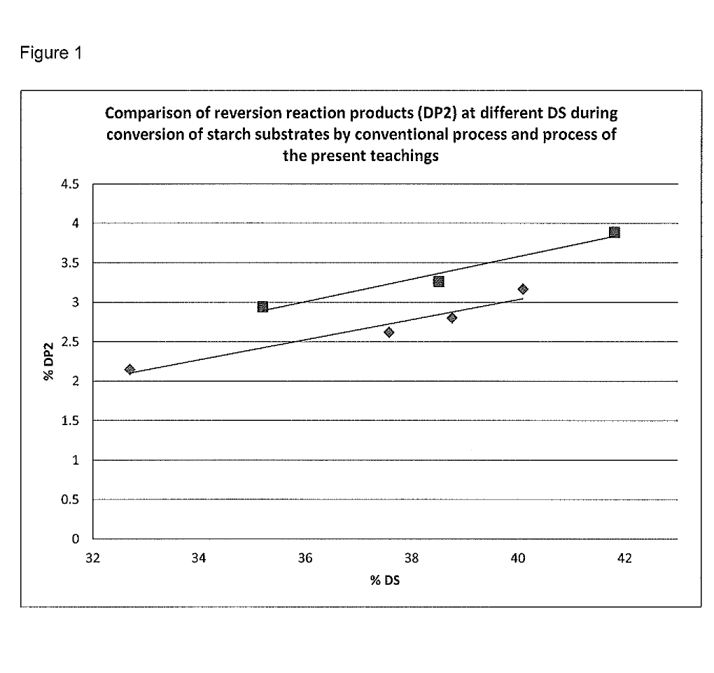

In an experiment typical of Example 1, the conventional process for starch hydrolysis was performed to illustrate results typical of the starch processing industry. Here, the liquefaction of the starch was carried out using an aqueous slurry of Cargill Gel™ 3240 unmodified dry corn starch at 35% dry solids content and the pH was adjusted to pH 5.6. Thermostable alpha-amylase, SPEZYME® FRED (Danisco-Genencor) was then added at 10 LU/gds starch and the starch slurry was pumped through a direct steam injection heater (jet-cooker) where the temperature was increased to 105±2° C. Exiting the jet cooker, the gelatinized starch was discharged into a pressurized primary liquefaction reactor and held for five minutes (105° C.) to completely gelatinize and solubilize the starch and to reduce the viscosity. From the primary liquefaction reactor, the soluble dextrin solution was discharged into a flash cooler to the secondary liquefaction temperature (95° C.) and pumped into a secondary liquefaction reactor. The hydrolysis was further continued for another 90 minutes and/or until a satisfactory DE (10-12 DE) was obtained. The residual alpha-amylase activity from the liquefaction step was inactivated by reducing the pH of the liquefact to pH 4.5 at 95° C. (held for 10 min) and used for saccharification studies. The DS of the liquefact was adjusted to 32% DS, 35% DS and 38% DS (concentrated by vacuum evaporation for higher DS) and the saccharification was carried out using the glucoamylase OPTIDEX® L-400 (Genencor-Danisco) at 0.20 GAU/gds starch, at pH 4.2 to 4.5 and 60° C. Samples were taken at different intervals of time and analyzed for sugar composition. Table 1 shows the effect of initial DS on DP2 content during hydrolysis of high temperature liquefied starch substrate by glucoamylase at pH 4.2 to pH 4.5, 60° C. For each of the three DS groups, the data for the highest glucose level (bold) are depicted in FIG. 1 as the top line (square legend).

TABLE 1

| | | | % composition of the sugar | | | |
|---|---|---|---|---|---|---|
| Initial | Final | Saccharification | | | | Hr. Sugar |
| DS | DS | Time, Hour | Glucose | DP2 | DP3 | (>DP3) |
| 32% | 35.2% | 24 | 91.90 | 1.90 | 0.82 | 5.38 |
| | | 47.5 | 94.98 | 2.50 | 0.54 | 1.98 |
| | | 63.5 | 95.57 | 2.94 | 0.46 | 1.03 |
| | | 71.5 | 95.28 | 3.09 | 0.43 | 1.20 |
| 35% | 38.5% | 24 | 90.75 | 2.00 | 0.50 | 6.74 |
| | | 47.5 | 94.28 | 2.76 | 0.38 | 2.57 |
| | | 63.5 | 95.00 | 3.26 | 0.39 | 1.35 |
| | | 71.5 | 94.82 | 3.48 | 0.40 | 1.31 |
| 38% | 41.8% | 24 | 89.60 | 2.25 | 0.56 | 7.58 |
| | | 47.5 | 93.54 | 3.08 | 0.47 | 2.91 |
| | | 63.5 | 94.01 | 3.65 | 0.49 | 1.85 |
| | | 71.5 | 94.13 | 3.89 | 0.50 | 1.49 |

### Example 2

The present teachings improve upon the conventional process presented in Example 1. For instance, Example 2 studies the effect of dry solids and DP2 during the hydrolysis of granular corn starch into high glucose syrup using an enzyme composition of an unexpectedly high dose alpha-amylase and low dose glucoamylase. In an experiment typical of Example 2, Cargill Gel™ 3240 unmodified dry corn starch slurry in distilled water was prepared containing different starting dry solids i.e., 32%, 38%, 41% and 43%. The pH of the slurry was then adjusted to pH 5.0, then alpha-amylase, SPEZYME® ALPHA at 10 AAU/gds, and glucoamylase, OPTIDEX® L-400 at 0.22 GAU/gds, were added and the slurry was placed in a water bath maintained at 60° C. The slurry was continuously stirred for uniform mixing during incubation. The samples were taken at different intervals of time during incubation, centrifuged to separate the undissolved starch. The clear supernatant was used to determine dissolved solids and sugar composition. The percent starch solubilized during incubation was also calculated.

Table 2 shows that for a given initial DS, the DP2 content increased with the increasing percent solubilization. The DP2 content in this granular starch hydrolysis is significantly lower than what is obtained in a conventional process at the same initial DS (see Table 1). This comparison is depicted in FIG. 1, which illustrates the lower reversion reaction product (DP2 formation) of the present teachings compared to the conventional two step process. Specifically, for each of the four DS groups in Table 2 below, the data for the 63 hour time point are depicted in FIG. 1 as the top line (diamond legend).

TABLE 2

| | Saccharifi- | | Super- | % composition of the sugar | | | |
|---|---|---|---|---|---|---|---|
| Initial | cation | | natant | | | | Hr. Sugar |
| DS | Time, Hour | % Sol. | DS | Glucose | DP2 | DP3 | (>DP3) |
| 32% | 23 | 79.2 | 30.6% | 92.40 | 2.05 | 1.71 | 3.84 |
| | 47 | 85.2 | 32.3% | 95.21 | 1.93 | 1.13 | 1.73 |
| | 63.5 | 86.6 | 32.7% | 95.90 | 2.15 | 0.81 | 1.13 |
| | 71 | 88.5 | 33.2% | 95.31 | 2.30 | 0.76 | 1.63 |
| 38% | 23 | 73.5 | 34.1% | 91.14 | 2.28 | 1.89 | 4.69 |
| | 47 | 82.2 | 37.0% | 93.81 | 2.36 | 1.40 | 2.43 |
| | 63 | 84.1 | 37.6% | 94.32 | 2.62 | 1.22 | 1.85 |
| | 70 | 85.1 | 37.9% | 94.77 | 2.61 | 1.08 | 1.54 |
| 41% | 23 | 71.0 | 35.4% | 91.84 | 2.35 | 1.82 | 3.98 |
| | 47 | 78.0 | 38.0% | 94.13 | 2.49 | 1.27 | 2.11 |
| | 63 | 80.1 | 38.8% | 94.62 | 2.80 | 1.05 | 1.53 |
| | 70 | 80.8 | 39.0% | 94.45 | 2.88 | 0.96 | 1.71 |
| 43% | 23 | 67.7 | 36.4% | 92.88 | 2.16 | 1.68 | 3.28 |
| | 47 | 74.8 | 39.2% | 94.41 | 2.68 | 1.13 | 1.78 |
| | 63 | 77.2 | 40.1% | 94.49 | 3.17 | 0.94 | 1.40 |
| | 70 | 78.6 | 40.6% | 94.52 | 3.30 | 0.87 | 1.31 |

### Example 3

This experiment shows the difference in DP2 between high and low activity of alpha-amylase during granular starch hydrolysis. Starch slurry was made to contain 38% and 35% dry substance starch and adjusted using the starch Be/DS tables (Corn Refiners Critical Data Tables pp 241-246). The native pH of the starch is 4.9-5.0 so no pH adjustments were required.

The dosing set up and sequence was as shown in Table 3a below.

TABLE 3a
Dose in Units/g of ds

| | AAU | GAU | | ASPU | |
|---|---|---|---|---|---|
| | (SPEZYME ® | (OPTIDEX ® | GAU | (OPTIMAX ® | |
| Test | XTRA) | L-400) | (H-GA) | L-1000) | % DS |
| 1 | 10 | 0.2 | | | 35 |
| 2 | 10 | 0.2 | | 0.15 | 35 |
| 3 | 10 | | 0.15 | | 35 |
| 4 | 10 | | 0.15 | 0.15 | 35 |
| 5 | 2 | | 0.3 | | 35 |
| 6 | 2 | 0.33 | | | 35 |
| 7 | 10 | 0.2 | | | 38 |
| 8 | 10 | 0.2 | | 0.15 | 38 |
| 9 | 10 | | 0.15 | | 38 |
| 10 | 10 | | 0.15 | 0.15 | 38 |
| 11 | 2 | | 0.3 | | 38 |
| 12 | 2 | 0.33 | | | 38 |

Starch slurries were dosed with enzymes and placed into 60° C. water baths equipped with 15 place submersible magnetic stirrers. Samples of the hydrolysis reaction were taken at different intervals of time during incubation and percent starch solubilization and the composition of the sugars were determined.

As seen in Table 3b below, higher level of alpha-amylase and lower glucoamylase dosed syrups contain significantly reduced amount of DP2 in 40 hours, resulting in similar levels of glucose as compared to lower alpha-amylase and higher glucoamylase treated syrups.

TABLE 3b

| | | | | | % | % Solubility | | | | | | Syrup % DS | | | | | |
|---|---|---|---|---|---|---|---|---|---|---|---|---|---|---|---|---|---|
| | AAU | AnGA | HGA | ASPU | DS | 16 hr | 24 | 40 | 48 | 64 | 88 | 16 hr | 24 | 40 | 48 | 64 | 88 |
| 1 | 10.0 | 0.2 | | | 35.0 | 70.1 | 75.2 | 81.1 | 81.0 | 83.5 | 84.5 | 30.4 | 32.0 | 33.6 | 33.8 | 34.6 | 34.8 |
| 2 | 10.0 | 0.2 | | 0.2 | 35.0 | 69.9 | 76.2 | 81.5 | 82.0 | 83.5 | 84.7 | 30.3 | 32.3 | 33.7 | 34.1 | 34.6 | 34.9 |
| 3 | 10.0 | | 0.2 | | 35.0 | 71.1 | 78.0 | 84.7 | 86.8 | 90.0 | 92.0 | 30.9 | 32.9 | 34.9 | 35.5 | 36.4 | 36.9 |
| 4 | 10.0 | | 0.2 | 0.2 | 35.0 | 71.5 | 78.9 | 86.2 | 88.2 | 90.8 | 93.0 | 31.0 | 33.2 | 35.3 | 35.9 | 36.6 | 37.2 |
| 5 | 2.0 | | 0.3 | | 35.0 | 75.5 | 82.0 | 88.4 | 90.2 | 92.4 | 93.8 | 32.2 | 34.1 | 35.9 | 36.4 | 37.0 | 37.4 |
| 6 | 2.0 | 0.3 | | | 35.0 | 64.5 | 70.6 | 74.2 | 76.5 | 78.3 | 79.9 | 28.7 | 30.7 | 31.8 | 32.5 | 33.1 | 33.5 |
| 7 | 10.0 | 0.2 | | | 38.0 | 64.8 | 71.4 | 75.5 | 77.0 | 79.1 | 80.2 | 31.8 | 34.0 | 35.3 | 35.8 | 36.5 | 36.8 |
| 8 | 10.0 | 0.2 | | 0.2 | 38.0 | 65.1 | 70.7 | 75.5 | 76.8 | 79.8 | 81.4 | 31.9 | 33.8 | 35.3 | 35.8 | 36.7 | 37.2 |
| 9 | 10.0 | | 0.2 | | 38.0 | 67.1 | 73.6 | 80.2 | 82.7 | 85.4 | 88.5 | 32.5 | 34.7 | 36.8 | 37.6 | 38.4 | 39.3 |
| 10 | 10.0 | | 0.2 | 0.2 | 38.0 | 67.4 | 74.1 | 80.7 | 83.2 | 86.3 | 89.0 | 32.7 | 34.9 | 37.0 | 37.7 | 38.6 | 39.4 |
| 11 | 2.0 | | 0.3 | | 38.0 | 71.8 | 76.8 | 83.0 | 85.0 | 89.0 | 89.0 | 33.9 | 35.8 | 37.7 | 38.3 | 39.4 | 39.4 |
| 12 | 2.0 | 0.3 | | | 38.0 | 62.0 | 67.2 | 72.4 | 73.9 | 75.5 | 77.4 | 30.8 | 32.6 | 34.3 | 34.8 | 35.3 | 35.9 |
| 13 | 10.0 | 0.2 | | 0.2 | 35.0 | 71.8 | 76.4 | 81.2 | 83.5 | 84.7 | 85.2 | 31.4 | 32.7 | 33.9 | 34.6 | 34.9 | 35.1 |
| 14 | 10.0 | | 0.2 | 0.2 | 35.0 | 76.5 | 82.1 | 88.2 | 89.6 | 91.4 | 93.0 | 32.5 | 34.2 | 35.9 | 36.3 | 36.8 | 37.2 |
| 15 | 10.0 | 0.2 | | 0.2 | 38.0 | 67.2 | 72.6 | 76.5 | 77.7 | 79.6 | 79.8 | 32.6 | 34.4 | 35.7 | 36.0 | 36.6 | 36.7 |
| 16 | 10.0 | | 0.2 | 0.2 | 38.0 | 71.4 | 76.5 | 82.1 | 84.8 | 86.6 | 88.3 | 34.0 | 35.7 | 37.4 | 38.2 | 38.8 | 39.2 |

| | % DP1 | | | | | | % DP2 | | | | | |
|---|---|---|---|---|---|---|---|---|---|---|---|---|
| | 16 hr | 24 | 40 | 48 | 64 | 88 | 16 hr | 24 | 40 | 48 | 64 | 88 |
| 1 | 90.1 | 92.7 | 94.7 | 95.1 | 95.4 | 95.7 | 2.6 | 2.2 | 2.1 | 2.2 | 2.5 | 2.9 |
| 2 | 91.7 | 94.0 | 95.6 | 95.9 | 96.0 | 95.9 | 3.1 | 2.4 | 2.1 | 2.2 | 2.5 | 2.9 |
| 3 | 90.6 | 93.7 | 95.6 | 95.5 | 95.8 | 95.4 | 3.6 | 2.7 | 2.6 | 2.8 | 3.2 | 3.8 |
| 4 | 91.1 | 94.2 | 95.8 | 95.7 | 95.8 | 95.5 | 4.4 | 3.0 | 2.6 | 2.7 | 3.1 | 3.6 |
| 5 | 96.2 | 96.3 | 95.5 | 94.9 | 94.2 | 93.1 | 2.3 | 2.8 | 3.8 | 4.3 | 5.1 | 6.0 |
| 6 | 94.2 | 95.1 | 95.9 | 96.0 | 96.0 | 95.6 | 1.9 | 2.1 | 2.4 | 2.5 | 2.9 | 3.4 |
| 7 | 90.6 | 92.7 | 94.6 | 95.0 | 95.3 | 95.2 | 2.5 | 2.3 | 2.3 | 2.4 | 2.7 | 3.2 |
| 8 | 92.4 | 94.3 | 95.7 | 95.8 | 95.8 | 95.4 | 2.9 | 2.4 | 2.3 | 2.5 | 2.8 | 3.4 |
| 9 | 92.2 | 94.3 | 95.5 | 95.4 | 95.3 | 94.7 | 3.1 | 2.7 | 2.9 | 3.2 | 3.7 | 4.4 |
| 10 | 92.1 | 94.5 | 95.7 | 95.8 | 95.4 | 94.8 | 3.9 | 2.9 | 2.9 | 3.0 | 3.5 | 4.1 |
| 11 | 96.1 | 96.0 | 94.9 | 94.4 | 93.3 | 92.0 | 2.5 | 3.1 | 4.3 | 4.9 | 5.8 | 6.9 |
| 12 | 94.4 | 95.2 | 95.6 | 95.7 | 95.6 | 95.0 | 2.1 | 2.3 | 2.7 | 2.9 | 3.4 | 4.0 |
| 13 | 89.9 | 93.1 | 95.2 | 95.6 | 95.8 | 95.9 | 4.1 | 2.7 | 2.1 | 2.1 | 2.3 | 2.6 |
| 14 | 93.6 | 95.4 | 96.0 | 95.9 | 95.3 | 94.6 | 3.1 | 2.6 | 2.9 | 3.2 | 3.8 | 4.5 |
| 15 | 90.7 | 93.4 | 95.3 | 95.5 | 95.7 | 95.6 | 3.7 | 2.6 | 2.2 | 2.3 | 2.6 | 2.9 |
| 16 | 93.9 | 95.3 | 95.7 | 95.5 | 94.9 | 94.0 | 3.0 | 2.8 | 3.2 | 3.5 | 4.2 | 5.0 |

### Example 4

In this example, a comparison of different commercially available alpha-amylases was made under high alpha-amylase and low glucoamylase conditions. The substrate was Cargill Gel™ 3240 unmodified dry corn starch. Commercial alpha-amylase dose was used at high doses of at least 3 to 4 times the dose recommended in the product technical product data sheet by the manufacturer in the context of the conventional two step process in the production of high glucose (e.g. the process depicted in Example 1). In an experiment typical of Example 4, to a 32% corn starch slurry at pH 5.0, alpha-amylase from the various different sources and glucoamylase, 0.22 GAU/gds of OPTIDEX® L-400 were added and incubated at 60° C., as explained in Example 2. Samples were taken at different intervals of time to determine the percent solubilization and final sugar composition. Data are presented in Table 4.

TABLE 4

| | | | | % composition of the sugar | | | |
|---|---|---|---|---|---|---|---|
| | Sacch. | | Supernatant | | | | Hr. Sugar |
| Alpha-amylase | Time, Hour | % Sol. | DS | Glucose | DP2 | DP3 | (>DP3) |
| 10 AAU/gs SPEZYME ® | 15.5 | 74.4 | 29.3 | 89.51 | 2.61 | 1.90 | 5.99 |
| XTRA | 23 | 79.2 | 30.6 | 92.40 | 2.05 | 1.71 | 3.84 |
| Bacillus | 47 | 85.2 | 32.3 | 95.21 | 1.93 | 1.13 | 1.73 |
| Stereothermophillus | 63.5 | 86.6 | 32.7 | 95.90 | 2.15 | 0.81 | 1.13 |
| 40 LU SPEZYME ® | 15.5 | 67.4 | 27.1 | 90.27 | 2.71 | 2.62 | 4.40 |
| FRED | 23 | 72.8 | 28.7 | 92.87 | 2.03 | 2.18 | 2.93 |
| Bacillus licheniformis | 47 | 77.2 | 30 | 95.53 | 1.94 | 1.22 | 1.32 |
| | 63.5 | 78.4 | 30.3 | 96.09 | 2.18 | 0.84 | 0.90 |
| 200MWU Fuelzyme ® LF | 15.5 | 55.5 | 23.2 | 89.48 | 2.77 | 2.67 | 5.07 |
| | 23 | 61.1 | 25 | 90.54 | 2.74 | 2.57 | 4.14 |
| | 47 | 76 | 29.5 | 90.44 | 3.52 | 2.78 | 3.27 |
| | 63.5 | 82.8 | 31.4 | 91.41 | 3.35 | 2.75 | 2.48 |
| 10 SSU GC626 | 15.5 | 26.3 | 12.3 | 97.47 | 1.19 | 0.00 | 1.34 |
| Aspergillus kawachii | 23 | 28.1 | 13.1 | 97.35 | 1.36 | 0.00 | 1.29 |
| | 47 | 31.2 | 14.3 | 96.55 | 1.92 | 0.12 | 1.41 |
| | 63.5 | 33.4 | 15.2 | 96.21 | 2.15 | 0.15 | 1.49 |
| 40 SKBU CLARASE L | 15.5 | 18.2 | 8.8 | 96.31 | 0.80 | 0.00 | 2.88 |
| Aspergillus oryzae | 23 | 19.5 | 9.4 | 96.28 | 1.04 | 0.00 | 2.67 |
| | 47 | 22.7 | 10.8 | 95.24 | 1.85 | 0.16 | 2.75 |
| | 63.5 | 24.9 | 11.8 | 93.93 | 2.73 | 0.19 | 3.14 |

Bacterial alpha-amylases successfully solubilized granular starch up to 86% while fungal alpha-amylases (GC626 and CLARASE L) were not as efficient.

### Example 5

In this example, a comparison of different commercially available glucoamylases was made under high alpha-amylase and low glucoamylase conditions. The substrate was granular corn starch. In an experiment typical of Example 5, 10 AAU/gds of SPEZYME® XTRA and one of three 3 different glucoamylases, 0.2 GAU/gds of OPTIDEX® L-400 (A. Niger glucoamylase, shown as An-GA), Humicola grisea glucoamylase), and Trichoderma reesei glucoamylase (Tr-GA, commercially known as GC321) were added to 32% corn starch slurry, pH 5.0, and incubated at 60° C., as explained in Example 2. Samples were taken at different intervals of time during incubation for determining the percent solubilization, final saccharide distribution, and other measures. Data are presented in Table 5. These data show that An-GA (OPTIDEX® L-400) produced lower levels of DP2 compared to H-GA and Tr-GA (GC321) at an equal dry solids, where>90% of granular starch was solubilized. These data also show that at all times points, H-GA is able to solubilize starch at higher levels than An-GA or Tr-GA.

TABLE 5

| | Sacch. | | Super- | % composition of the sugar | | | |
|---|---|---|---|---|---|---|---|
| | Time, | % | natant | | | | Hr. Sugar |
| glucoamylase | Hour | Sol. | DS | Glucose | DP2 | DP3 | (>DP3) |
| An-GA or | 16 | 79.0 | 29.5 | 82.79 | 5.01 | 2.12 | 10.08 |
| OPTIDEX ® | 24.5 | 85.6 | 30.4 | 88.44 | 2.71 | 2.22 | 6.64 |
| L-400 | 48 | 91.1 | 32.7 | 92.83 | 1.86 | 1.80 | 3.52 |
| | 71.5 | 92.9 | 33.1 | 93.97 | 2.04 | 1.37 | 2.61 |
| H-GA | 16 | 86.1 | 31.4 | 93.21 | 2.46 | 1.29 | 3.04 |
| | 24.5 | 92.7 | 32.2 | 94.53 | 2.54 | 0.92 | 2.01 |
| | 48 | 97.4 | 34.2 | 94.86 | 3.44 | 0.60 | 1.10 |
| | 71.5 | 98.1 | 34.4 | 94.36 | 4.19 | 0.56 | 0.89 |
| Tr-GA | 16 | 78.4 | 29.3 | 81.10 | 5.94 | 1.49 | 11.47 |
| Or GC321 | 24.5 | 82.4 | 30.0 | 83.75 | 4.74 | 1.47 | 10.04 |
| | 48 | 88.5 | 32.0 | 90.94 | 2.73 | 1.01 | 5.32 |
| | 71.5 | 90.0 | 32.4 | 92.86 | 2.95 | 0.75 | 3.45 |

### Example 6

### Effect of Glucoamylase Blending

In this example, enzyme blends with An-GA and H-GA at different ratios were prepared and applied under high alpha-amylase (10 AAU/gds of SPEZYME® XTRA) and low glucoamylase conditions. The substrate was granular corn starch. The total glucoamylase dose used was 0.18 GAU/gds, while 5 different ratios of An-GA:H-GA were used (100:0, 75:25, 50:50, 25:75 and 0:100), using 35% DS aqueous corn starch slurry, pH 5.0, and 32% DS aqueous corn starch slurry and incubated at 60° C. as explained in Example 2. Samples were taken at different intervals of time during incubation for determining the percent solubilization, final glucose composition, and other measures. Data are presented in Table 6. Blending An-GA with H-GA at least 25% on an activity (GAU) basis resulted in significantly reduced DP2 at the same % dry solids, maintaining 95.5%>DP1. Bold numbers indicate DP2 levels for comparison at an equal % DS (85-86%).

TABLE 6

| An-GA: | Sacch. | | | % composition of the sugar | | | |
|---|---|---|---|---|---|---|---|
| H-GA | Time, | % | Supernatant | | | | Hr. Sugar |
| ratio | Hour | Sol. | DS | Glucose | DP2 | DP3 | (>DP3) |
| 100:0 | 17 | 69.6 | 29.7 | 90.63 | 1.57 | 2.04 | 5.76 |
| | 24 | 75.0 | 31.4 | 91.90 | 1.69 | 1.93 | 4.48 |
| | 48 | 82.6 | 33.6 | 94.85 | 1.70 | 1.17 | 2.28 |
| | 70 | 86.1 | 34.7 | 95.36 | 2.09 | 0.87 | 1.68 |
| 75:25 | 17 | 71.5 | 31.2 | 92.14 | 1.80 | 1.75 | 4.31 |
| | 24 | 77.1 | 33.0 | 93.30 | 1.62 | 1.60 | 3.48 |
| | 48 | 83.5 | 34.9 | 95.72 | 2.21 | 0.81 | 1.25 |
| | 70 | 86.2 | 35.8 | 95.61 | 2.57 | 0.61 | 1.22 |
| 50:50 | 17 | 72.1 | 31.6 | 92.95 | 1.78 | 1.53 | 3.74 |
| | 24 | 77.5 | 33.4 | 94.54 | 1.72 | 1.25 | 2.49 |
| | 48 | 84.4 | 35.5 | 95.75 | 2.29 | 0.66 | 1.30 |
| | 70 | 89.0 | 36.9 | 95.12 | 3.11 | 0.55 | 1.22 |
| 25:75 | 17 | 73.4 | 32.0 | 94.84 | 1.49 | 1.07 | 2.60 |
| | 24 | 78.9 | 33.7 | 95.20 | 1.96 | 0.86 | 1.99 |
| | 48 | 86.3 | 36.0 | 95.85 | 2.65 | 0.51 | 0.99 |
| | 70 | 89.6 | 37.0 | 95.49 | 3.28 | 0.45 | 0.79 |
| 0:100 | 17 | 73.8 | 32.5 | 93.97 | 2.23 | 1.14 | 2.67 |
| | 24 | 79.0 | 34.2 | 94.57 | 2.48 | 0.94 | 2.01 |
| | 48 | 86.0 | 36.3 | 95.63 | 2.76 | 0.56 | 1.06 |
| | 70 | 90.0 | 37.5 | 94.80 | 3.48 | 0.52 | 1.20 |

### Example 7

### Effect of Temperature Staging on Reversion Reaction

In the direct starch to glucose process reversion reaction occurs due to formation of oligosaccharides (mainly DP2) from glucose by the glucoamylase. This reversion reaction is unwanted, as it lowers the DP1 concentration and creates unwanted byproducts. As the rate of the reversion reaction is dependent on glucose concentration, it is mainly observed at high solubilities (>85%) and also increasing with increased initial DS. The purpose of this experiment is to see if we could reduce the reversion reaction by increasing the temperature after 30 hours. Temperature was increased from 60° C. to 66° C., as this is supposed to (partially) inactivate the glucoamylase, thus reducing or stopping the (reversion) activity of the glucoamylase.

The total glucoamylase dose used was 0.15 GAU/gds for H-GA and 0.18 GAU/gds for An-GA. The 55:45 blend of both enzymes contained 0.075 GAU/gds of H-GA and 0.09 GAU/gds of An-GA (total 0.165 GAU/gds). A 32% DS corn starch slurry was prepared using tap water and dry bag starch from Roquette obtained via Barentz. Slurry was incubated at 60° C. at pH 4.9, 10 AAU/gds SPEZYME® XTRA and the before mentioned doses of glucoamylase. Samples were taken at different time intervals during incubation for determining the percent solubilization and sugar composition.

Table 7A contains data of the experiment where the temperature was increased from 60° C. to 66° C. after 30 hours. Reference table for this experiment contains data of an experiment where temperature was maintained at 60° C. throughout the complete hydrolysis (Table 7B). Italic numbers in Table 7A display values that are lower than the reference values, bold numbers display values that are higher than the reference. Displayed are average values and standard deviations of duplicate incubations in one experiment.

TABLE 7A

| An-GA: | Sacch. | | | % composition of the sugar | | | |
|---|---|---|---|---|---|---|---|
| H-GA | Time, | | Supernatant | | | | Hr. Sugar |
| ratio | Hour | % Sol. | DS | Glucose | DP2 | DP3 | (>DP3) |
| 100:0 | 22.5 | 82.9 ± 0.2 | 31.4 ± 0.1 | 90.99 ± 0.05 | 2.11 ± 0.01 | 2.48 ± 0.04 | 4.42 ± 0.00 |
| | 30 | 87.0 ± 0.6 | 32.6 ± 0.0 | 92.73 ± 0.00 | 2.01 ± 0.01 | 1.72 ± 0.01 | 3.54 ± 0.02 |
| | 47 | 91.5 ± 0.6 | 33.9 ± 0.0 | 93.36 ± 0.11 | 2.26 ± 0.05 | 1.54 ± 0.01 | 2.84 ± 0.05 |
| | 54 | 93.0 ± 0.3 | 34.3 ± 0.1 | 93.33 ± 0.31 | 2.37 ± 0.16 | 1.53 ± 0.03 | 2.76 ± 0.12 |
| | 71 | 93.7 ± 0.9 | 34.5 ± 0.1 | 93.46 ± 0.22 | 2.37 ± 0.14 | 1.56 ± 0.01 | 2.61 ± 0.07 |
| 55:45 | 22.5 | 84.6 ± 0.7 | 32.1 ± 0.3 | 92.91 ± 0.11 | 2.18 ± 0.04 | 2.00 ± 0.00 | 2.90 ± 0.07 |
| | 30 | 89.0 ± 0.7 | 33.4 ± 0.3 | 94.51 ± 0.09 | 2.18 ± 0.01 | 1.19 ± 0.03 | 2.13 ± 0.05 |
| | 47 | 92.9 ± 0.4 | 34.5 ± 0.2 | 94.74 ± 0.05 | 2.58 ± 0.01 | 0.99 ± 0.01 | 1.69 ± 0.03 |
| | 54 | 93.6 ± 0.7 | 34.7 ± 0.3 | 94.92 ± 0.07 | 2.42 ± 0.01 | 1.00 ± 0.03 | 1.66 ± 0.02 |
| | 71 | 94.7 ± 0.7 | 35.0 ± 0.3 | 94.89 ± 0.10 | 2.39 ± 0.01 | 1.07 ± 0.04 | 1.65 ± 0.04 |
| 0:100 | 22.5 | 82.5 ± 0.6 | 31.7 ± 0.2 | 94.45 ± 0.05 | 2.19 ± 0.00 | 1.71 ± 0.02 | 1.65 ± 0.03 |
| | 30 | 87.5 ± 0.6 | 33.2 ± 0.3 | 96.07 ± 0.18 | 2.10 ± 0.05 | 0.73 ± 0.12 | 1.10 ± 0.02 |
| | 47 | 91.8 ± 0.8 | 34.4 ± 0.3 | 95.64 ± 0.10 | 2.76 ± 0.02 | 0.66 ± 0.03 | 0.94 ± 0.06 |
| | 54 | 92.3 ± 0.4 | 34.6 ± 0.4 | 95.55 ± 0.08 | 2.84 ± 0.02 | 0.65 ± 0.01 | 0.95 ± 0.04 |
| | 71 | 92.9 ± 0.5 | 34.7 ± 0.3 | 95.64 ± 0.04 | 2.68 ± 0.00 | 0.68 ± 0.00 | 1.00 ± 0.04 |

TABLE 7B

| An-GA: | Sacch. | | | % composition of the sugar | | | |
|---|---|---|---|---|---|---|---|
| H-GA | Time, | | Supernatant | | | | Hr. Sugar |
| ratio | Hour | % Sol. | DS | Glucose | DP2 | DP3 | (>DP3) |
| 100:0 | 22.5 | 82.4 ± 0.3 | 31.6 ± 0.1 | 90.95 ± 0.17 | 2.14 ± 0.04 | 2.46 ± 0.01 | 4.45 ± 0.12 |
| | 30 | 86.8 ± 0.4 | 32.8 ± 0.1 | 92.73 ± 0.19 | 2.00 ± 0.06 | 1.70 ± 0.01 | 3.56 ± 0.12 |
| | 47 | 90.0 ± 0.1 | 33.8 ± 0.0 | 94.61 ± 0.00 | 1.79 ± 0.03 | 1.41 ± 0.02 | 2.19 ± 0.02 |
| | 54 | 90.9 ± 0.0 | 34.0 ± 0.0 | 94.94 ± 0.02 | 1.88 ± 0.03 | 1.28 ± 0.01 | 1.90 ± 0.04 |
| | 71 | 92.4 ± 0.3 | 34.5 ± 0.1 | 95.56 ± 0.09 | 1.90 ± 0.00 | 1.08 ± 0.02 | 1.46 ± 0.07 |
| 55:45 | 22.5 | 85.0 ± 0.1 | 32.0 ± 0.1 | 93.24 ± 0.13 | 2.10 ± 0.01 | 1.98 ± 0.01 | 2.67 ± 0.11 |
| | 30 | 89.2 ± 0.4 | 33.2 ± 0.0 | 94.72 ± 0.12 | 2.16 ± 0.00 | 1.14 ± 0.03 | 1.97 ± 0.09 |
| | 47 | 93.6 ± 0.2 | 34.5 ± 0.2 | 95.95 ± 0.08 | 2.24 ± 0.02 | 0.80 ± 0.03 | 1.01 ± 0.07 |
| | 54 | 94.5 ± 0.2 | 34.7 ± 0.2 | 96.12 ± 0.07 | 2.36 ± 0.03 | 0.71 ± 0.04 | 0.81 ± 0.06 |
| | 71 | 96.3 ± 0.0 | 35.2 ± 0.1 | 96.37 ± 0.02 | 2.55 ± 0.05 | 0.56 ± 0.02 | 0.51 ± 0.05 |
| 0:100 | 22.5 | 83.5 ± 0.5 | 31.8 ± 0.1 | 94.48 ± 0.16 | 2.20 ± 0.02 | 1.62 ± 0.16 | 1.70 ± 0.02 |
| | 30 | 88.2 ± 0.4 | 33.1 ± 0.1 | 95.25 ± 0.03 | 2.24 ± 0.01 | 1.43 ± 0.01 | 1.09 ± 0.02 |
| | 47 | 92.6 ± 0.3 | 34.4 ± 0.0 | 96.25 ± 0.03 | 2.68 ± 0.02 | 0.54 ± 0.00 | 0.53 ± 0.01 |
| | 54 | 93.9 ± 0.2 | 34.8 ± 0.1 | 96.12 ± 0.01 | 3.00 ± 0.01 | 0.50 ± 0.03 | 0.38 ± 0.01 |
| | 71 | 95.6 ± 0.4 | 35.2 ± 0.2 | 96.04 ± 0.03 | 3.26 ± 0.02 | 0.45 ± 0.00 | 0.25 ± 0.01 |

In the experiments containing H-GA alone, the DP2 indeed can be reduced by increasing the temperature from 60° C. to 66° C. However, this is at the expense of both solubilization and DP1. On the other hand, when An-GA (either alone or in a blend with H-GA) is present, the temperature staging does not result in lowered DP2 (only at 71 hours for the blend). Interestingly enough, An-GA as single enzyme even shows higher solubilities upon temperature staging, indicating that An-GA is more thermostable than H-GA. At this increased temperature An GA even outperforms H-GA with regards to solubilization, albeit that DP1 is low.

### Example 8

### Effect of Pullulanase Addition

In conventional processes, debranching enzymes such as pullulanases are used to increase DP1 concentrations. To see if debranching enzymes could elevate the DP1 concentrations during granular starch hydrolysis of corn starch, pullulanase OPTIMAX® L-1000 was added and its effect on solubilization and sugar profile was measured. A 32% DS corn starch slurry was prepared using dry bag starch from Roquette and tap water, adjusting the pH to 4.9. The slurry was divided over Schott Duran bottles, preparing all experiments in duplicate, and enzymes were added to the bottles. Pullulanase addition was applied at varying doses of 0, 0.125, 0.5, 1.0, and 3.0 ASPU/gds, while maintaining the alpha-amylase and glucoamylase doses constant at 10 AAU/gds SPEZYME® XTRA and 0.15 GAU/gds H-GA, respectively. After enzyme addition, the flasks were incubated at 60° C. Samples were taken at different time intervals during incubation for determining the percent solubilization and sugar composition.

Displayed in Table 8 are average values and standard deviation measures of duplicate incubations in one experiment. Italic numbers in Table 8 display values that are lower than the reference values of the experiment without pullulanase addition at the same time point, bold numbers display values that are higher than the reference for that time.

Table 8 demonstrates that both the solubilization% and the DP1% levels increase at increasing pullulanase concentrations, which improves glucose concentrations compared to the reference experiment. The increase in glucose concentration is the result from hydrolysis of the oligosaccharides, as can be seen by the decreased oligosaccharide DP3 and DP3+ levels (Table 8).

TABLE 8

| OPTIMAX ® | | | | | | | |
|---|---|---|---|---|---|---|---|
| L-1000 | Sacch. | | | % composition of the sugar | | | |
| dosage | Time, | | Supernatant | | | | Hr. Sugar |
| (ASPU/gds) | Hour | % Sol. | DS | Glucose | DP2 | DP3 | (>DP3) |
| 0 | 5 | 62.20 ± 0.12 | 25.03 ± 0.02 | 76.28 ± 0.55 | 10.43 ± 0.45 | 1.35 ± 0.05 | 11.94 ± 0.15 |
| | 22.5 | 84.54 ± 0.11 | 31.97 ± 0.01 | 94.16 ± 0.23 | 2.40 ± 0.03 | 1.05 ± 0.05 | 2.40 ± 0.14 |
| | 29 | 87.17 ± 0.13 | 32.74 ± 0.01 | 94.97 ± 0.20 | 2.38 ± 0.01 | 0.86 ± 0.05 | 1.79 ± 0.14 |
| | 46.5 | 92.60 ± 0.18 | 34.29 ± 0.08 | 95.79 ± 0.03 | 2.70 ± 0.04 | 0.56 ± 0.02 | 0.95 ± 0.05 |
| | 53 | 93.09 ± 0.27 | 34.43 ± 0.10 | 95.89 ± 0.06 | 2.83 ± 0.03 | 0.50 ± 0.02 | 0.79 ± 0.07 |
| 0.125 | 5 | 63.18 ± 0.75 | 25.33 ± 0.25 | 76.93 ± 0.02 | 11.52 ± 0.03 | 1.64 ± 0.001 | 9.91 ± 0.01 |
| | 22.5 | 85.43 ± 0.03 | 32.21 ± 0.01 | 94.75 ± 0.06 | 2.36 ± 0.13 | 1.09 ± 0.03 | 1.80 ± 0.16 |
| | 29 | 88.3 ± 0.54 | 33.05 ± 0.15 | 95.29 ± 0.03 | 2.42 ± 0.003 | 0.85 ± 0.02 | 1.44 ± 0.02 |
| | 46.5 | 93.6 ± 0.14 | 34.55 ± 0.04 | 95.81 ± 0.05 | 2.74 ± 0.03 | 0.52 ± 0.005 | 0.93 ± 0.01 |
| | 53 | 94.0 ± 0.14 | 34.68 ± 0.04 | 95.76 ± 0.01 | 2.89 ± 0.01 | 0.47 ± 0.002 | 0.88 ± 0.03 |
| 0.5 | 5 | 62.5 ± 0.21 | 25.13 ± 0.06 | 78.35 ± 0.08 | 13.12 ± 0.13 | 1.93 ± 0.002 | 6.60 ± 0.05 |
| | 22.5 | 85.9 ± 0.18 | 32.36 ± 0.04 | 95.30 ± 0.07 | 2.47 ± 0.02 | 1.01 ± 0.01 | 1.22 ± 0.04 |
| | 29 | 89.17 ± 0.49 | 33.29 ± 0.15 | 95.75 ± 0.02 | 2.41 ± 0.02 | 0.77 ± 0.01 | 1.07 ± 0.01 |
| | 46.5 | 94.03 ± 0.35 | 34.67 ± 0.09 | 95.97 ± 0.01 | 2.75 ± 0.0002 | 0.48 ± 0.004 | 0.79 ± 0.003 |
| | 53 | 94.60 ± 0.16 | 34.83 ± 0.04 | 95.94 ± 0.07 | 2.89 ± 0.03 | 0.44 ± 0.02 | 0.72 ± 0.03 |
| 1.0 | 5 | 62.91 ± 0.56 | 25.28 ± 0.21 | 78.54 ± 0.18 | 14.58 ± 0.13 | 2.08 ± 0.03 | 4.80 ± 0.07 |
| | 22.5 | 86.26 ± 0.13 | 32.50 ± 0.000 | 95.68 ± 0.03 | 2.46 ± 0.01 | 0.94 ± 0.0001 | 0.93 ± 0.04 |
| | 29 | 89.66 ± 0.77 | 33.48 ± 0.26 | 96.07 ± 0.03 | 2.39 ± 0.02 | 0.72 ± 0.01 | 0.82 ± 0.02 |
| | 46.5 | 94.26 ± 0.25 | 34.78 ± 0.03 | 96.13 ± 0.06 | 2.76 ± 0.03 | 0.46 ± 0.01 | 0.65 ± 0.02 |
| | 53 | 94.86 ± 0.18 | 34.95 ± 0.09 | 96.04 ± 0.01 | 2.92 ± 0.01 | 0.43 ± 0.0003 | 0.61 ± 0.02 |
| 3.0 | 5 | 63.33 ± 0.33 | 25.31 ± 0.05 | 78.77 ± 0.54 | 16.43 ± 0.50 | 2.03 ± 0.01 | 2.77 ± 0.03 |
| | 22.5 | 86.92 ± 0.46 | 32.55 ± 0.08 | 95.91 ± 0.19 | 2.49 ± 0.05 | 0.86 ± 0.07 | 0.74 ± 0.08 |
| | 29 | 90.16 ± 1.00 | 33.48 ± 0.06 | 96.63 ± 0.46 | 2.41 ± 0.003 | 0.65 ± 0.02 | 0.31 ± 0.44 |
| | 46.5 | 94.75 ± 0.39 | 34.77 ± 0.12 | 96.77 ± 0.02 | 2.78 ± 0.04 | 0.46 ± 0.02 | 0 |
| | 53 | 95.41 ± 0.54 | 34.95 ± 0.08 | 96.68 ± 0.01 | 2.91 ± 0.02 | 0.41 ± 0.01 | 0 |

### Example 9

### Effect of Enzyme Staging

In a hydrolysis reaction where all enzymes are added in the beginning of starch hydrolysis, DP1 concentration will increase fast, while solubilization % will progress slowly. This results in a fast back reaction of DP1, converting DP1 into, for example, isomaltose and increasing the less favorable DP2 concentration. By delaying the addition of some of the enzymes, we aimed to postpone the DP1 maximum and, thus, reduce the reversion reaction into DP2. Therefore, the effect of delayed enzyme addition, or also called enzyme staging, was investigated on solubilization and sugar composition with the purpose to synchronize solubilization with DP1. In this example, a strategy of delayed addition of alpha-amylase and/or glucoamylase at different time points is under investigation.

A 32% DS corn starch slurry was prepared using dry bag starch from Roquette and tap water, adjusting the pH to 4.9. The slurry was divided over scott bottles, preparing all experiments in duplicates, and part of the enzymes were added. A first dose of 2 AAU/gds SPEZYME® XTRA was added from the start of hydrolysis; and a second dose of 2 AAU/gds SPEZYME® XTRA and/or H-GA at 0.25 GAU/gds were added at 0, 20 or 44 hrs, as indicated in Table 9. After the first enzyme addition, the flasks were incubated at 60° C. Samples were taken at different time intervals during incubation for determining the percent solubilization and sugar composition.

Displayed in Table 9 are average values and standard deviation measured of duplicate incubations in one experiment. Bold underlined numbers in Table 9 display the DP1 maximum values of each experiment, and italic underlined numbers display the DP2 minimum values. The data in Table 9 demonstrate that delaying part of the alpha-amylase addition to 20 hrs (experiment #2) or delaying part of the alpha-amylase and glucoamylase to 20 hrs (experiment #3) significantly increases the solubilization% values to ˜94.8-97.2% after 51 hrs (see control experiment #1 for comparison).

Furthermore, synchronization of DP1%, DP2% and solubilization% values are observed when the addition of glucoamylase is postponed (as can be seen in Experiments #3, 4 and 5 in Table 9). By postponing the glucoamylase addition, the DP1% maximum and DP2% minimum are delayed and consequently synchronized with improved solubilization % values.

To conclude, delayed addition of part of the alpha-amylase results in high solubilization, which may be related to enzyme stability or substrate inhibition. Delayed addition of glucoamylase leads to synchronization of solubilization and DP1 values and reduces DP2 concentration, since the DP1 formation reaction is postponed, delaying the reversion reaction. These results show that both enzymes alpha-amylase and glucoamylase are more efficient when they are (partially) staged, resulting in improved process results.

TABLE 9

| | Sacch. | | | % composition of the sugar | | | |
|---|---|---|---|---|---|---|---|
| | Time, | | Supernatant | | | | Hr. Sugar |
| Dosages added | Hour | % Sol. | DS | Glucose | DP2 | DP3 | (>DP3) |
| Experiment #1 | 4 | 61.15 ± 0.78 | 23.83 ± 0.01 | 88.09 ± 0.29 | 4.09 ± 0.15 | 0.98 ± 0.01 | 6.84 ± 0.15 |
| At 0 hrs: | 20 | 86.29 ± 0.99 | 31.34 ± 0.07 | 96.10 ± 0.04 | 2.26 ± 0.03 | 0.47 ± 0.001 | 1.18 ± 0.07 |
| SPEZYME ® XTRA = 4 AAU/gds | 28 | 91.29 ± 0.86 | 32.72 ± 0.13 | 96.16 ± 0.05 | 2.61 ± 0.01 | 0.38 ± 0.005 | 0.86 ± 0.06 |
| H-GA = 0.25 GAU/gds | 44 | 94.83 ± 1.46 | 33.68 ± 0.76 | 95.65 ± 0.01 | 3.38 ± 0.02 | 0.97 ± 0.005 | — |
| | 51 | 92.75 ± 1.03 | 33.12 ± 0.64 | 95.37 ± 0.11 | 3.70 ± 0.08 | 0.67 ± 0.34 | 0.26 ± 0.37 |
| | 68 | 91.41 ± 1.13 | 32.76 ± 0.67 | 94.72 ± 0.11 | 4.28 ± 0.11 | 1.00 ± 0.003 | — |
| | 75 | 91.93 ± 0.77 | 32.90 ± 0.57 | 94.64 ± 0.11 | 4.41 ± 0.04 | 0.95 ± 0.03 | — |
| Experiment #2 | 4 | 57.44 ± 1.29 | 22.91 ± 0.12 | 90.05 ± 0.16 | 3.06 ± 0.01 | 0.76 ± 0.02 | 6.13 ± 0.14 |
| At 0 hrs: | 20 | 80.90 ± 0.96 | 30.20 ± 0.69 | 96.39 ± 0.02 | 2.19 ± 0.07 | 0.35 ± 0.01 | 1.07 ± 0.08 |
| SPEZYME ® XTRA = 2 AAU/gds | 28 | 88.08 ± 0.45 | 32.25 ± 0.32 | 96.07 ± 0.001 | 2.62 ± 0.05 | 0.35 ± 0.01 | 0.97 ± 0.04 |
| H-GA = 0.25 GAU/gds | 44 | 93.79 ± 0.25 | 33.83 ± 0.40 | 95.62 ± 0.07 | 3.35 ± 0.05 | 1.03 ± 0.02 | — |
| At 20 hrs: | 51 | 93.49 ± 1.48 | 33.75 ± 0.87 | 95.21 ± 0.35 | 3.66 ± 0.10 | 1.13 ± 0.25 | — |
| SPEZYME ® XTRA = 2 AAU/gds | 68 | 94.76 ± 2.59 | 34.10 ± 1.18 | 94.78 ± 0.06 | 4.23 ± 0.03 | 0.99 ± 0.03 | — |
| | 75 | 95.13 ± 2.56 | 34.20 ± 1.17 | 94.61 ± 0.12 | 4.43 ± 0.08 | 0.97 ± 0.04 | — |
| Experiment #3 | 4 | 34.08 ± 0.21 | 14.66 ± 0.01 | 1.14 ± 0.02 | 9.00 ± 0.003 | 11.47 ± 0.30 | 78.38 ± 0.32 |
| At 0 hrs: | 20 | 44.37 ± 0.47 | 18.50 ± 0.29 | 1.76 ± 0.38 | 9.53 ± 1.37 | 12.24 ± 0.89 | 76.47 ± 2.64 |
| SPEZYME ® XTRA = 2 AAU/gds | 28 | 72.63 ± 0.55 | 27.87 ± 0.02 | 93.25 ± 0.27 | 2.17 ± 0.02 | 1.21 ± 0.06 | 3.36 ± 0.18 |
| At 20 hrs: | 44 | 82.29 ± 0.77 | 32.75 ± 0.004 | 96.05 ± 0.01 | 2.59 ± 0.01 | 0.41 ± 0.0004 | 0.95 ± 0.001 |
| SPEZYME ® XTRA = 2 AAU/gds | 51 | 92.73 ± 0.55 | 33.71 ± 0.07 | 95.88 ± 0.01 | 2.98 ± 0.04 | 0.40 ± 0.01 | 0.74 ± 0.02 |
| H-GA = 0.25 GAU/gds | 68 | 96.18 ± 0.11 | 34.66 ± 0.20 | 95.14 ± 0.14 | 3.89 ± 0.05 | 0.97 ± 0.08 | — |
| | 75 | 97.20 ± 0.12 | 34.94 ± 0.20 | 94.89 ± 0.10 | 4.20 ± 0.05 | 0.91 ± 0.05 | — |
| Experiment #4 | 4 | 34.00 ± 0.11 | 14.64 ± 0.14 | 1.18 ± 0.05 | 8.95 ± 0.06 | 11.36 ± 0.07 | 78.51 ± 0.17 |
| At 0 hrs: | 20 | 44.87 ± 0.73 | 18.69 ± 0.39 | 1.89 ± 0.14 | 9.81 ± 0.39 | 12.23 ± 0.48 | 76.07 ± 1.01 |
| SPEZYME ® XTRA = 2 AAU/gds | 28 | 48.78 ± 1.35 | 20.08 ± 0.61 | 2.53 ± 0.05 | 10.95 ± 0.07 | 13.57 ± 0.05 | 72.95 ± 0.07 |
| At 20 hrs: | 44 | 53.82 ± 1.82 | 21.82 ± 0.77 | 3.22 ± 0.08 | 11.69 ± 0.09 | 14.59 ± 0.07 | 70.51 ± 0.24 |
| SPEZYME ® XTRA = 2 AAU/gds | 51 | 72.18 ± 1.01 | 27.75 ± 0.50 | 91.88 ± 0.77 | 2.59 ± 0.31 | 1.67 ± 0.11 | 3.86 ± 0.35 |
| At 44 hrs: | 68 | 90.86 ± 0.60 | 33.21 ± 0.40 | 96.04 ± 0.07 | 2.67 ± 0.01 | 0.43 ± 0.03 | 0.86 ± 0.02 |
| H-GA = 0.25 GAU/g ds | 75 | 93.90 ± 0.67 | 34.05 ± 0.42 | 95.81 ± 0.04 | 3.13 ± 0.005 | 0.40 ± 0.01 | 0.66 ± 0.04 |
| Experiment #5 | 4 | 33.83 ± 0.53 | 14.71 ± 0.29 | 1.12 | 8.73 | 11.34 | 78.81 |
| At 0 hrs: | 20 | 45.02 ± 0.98 | 18.92 ± 0.47 | 1.86 ± 0.03 | 9.88 ± 0.22 | 12.36 ± 0.24 | 75.90 ± 0.50 |
| SPEZYME ® XTRA = 2 AAU/gds | 28 | 47.95 ± 1.70 | 19.97 ± 0.72 | 2.13 ± 0.01 | 10.45 ± 0.26 | 13.03 ± 0.20 | 74.38 ± 0.45 |
| At 44 hrs: | 44 | 52.28 ± 1.96 | 21.50 ± 0.81 | 2.51 ± 0.05 | 10.75 ± 0.03 | 13.37 ± 0.04 | 73.38 ± 0.02 |
| SPEZYME ® XTRA = 2 AAU/gds | 51 | 72.29 ± 0.99 | 28.05 ± 0.47 | 91.85 ± 0.36 | 2.59 ± 0.14 | 1.54 ± 0.07 | 4.02 ± 0.15 |
| H-GA = 0.25 GAU/g ds | 68 | 89.76 ± 0.61 | 33.23 ± 0.38 | 96.24 ± 0.26 | 2.62 ± 0.11 | 0.37 ± 0.08 | 0.78 ± 0.07 |
| | 75 | 92.65 ± 0.76 | 34.04 ± 0.42 | 95.81 ± 0.08 | 3.10 ± 0.05 | 0.40 ± 0.01 | 0.68 ± 0.03 |

### Example 10

### Effect of Glucoamylase Staging

Example 9 showed the possibility to synchronize solubilization and DP1 values by delayed addition of enzymes. The purpose of Example 10 is to better understand the optimal time for glucoamylase staging.

A 32% DS corn starch slurry was prepared using dry bag starch from Roquette and tap water, adjusting the pH to 4.9. The slurry was divided over Schott Duran bottles, preparing all experiments in duplicate, and part of the enzymes were added. All SPEZYME® XTRA dosage of 10 AAU/gds was added from the start of hydrolysis; and H-GA addition of 0.15 GAU/gds was delayed at 0, 5 or 10 hrs, as indicated in Table 10. After the alpha-amylase addition, the flasks were incubated at 60° C. Samples were taken at different time intervals during incubation for determining the percent solubilization and sugar composition.

Displayed in Table 10 are average values and standard deviation measures of duplicate incubations in one experiment. Bold underlined numbers in Table 10 display the DP1 maximum values of each experiment, and italic underlined numbers display the DP2 minimum values. By staging the H-GA, the DP1% maximum and DP2% minimum are postponed, as can be seen for Experiment #3 with H-GA addition at 10 hrs compared to the reference experiment #1 without enzyme staging. By postponing the DP1% peak, synchronization occurs between high solubilization levels, increased DP1 values, and decreased DP2 values. In addition, Experiment #2 demonstrates that, for the chosen reaction conditions, enzyme staging at 5 hrs does not influence the DP1 or DP2 values, but does significantly increase the solubilization % after 48 hrs. This result indicates that when H-GA is added from the start of starch hydrolysis, some of the enzyme activity is lost due to enzyme inactivation or inhibition. These results confirm those observed in Example 9 that enzyme staging is a beneficial strategy to optimize solubilization, DP1 and DP2 levels.

TABLE 10

| | Sacch. | | | % composition of the sugar | | | |
|---|---|---|---|---|---|---|---|
| H-GA dosage | Time, | | Supernatant | | | | Hr. Sugar |
| (GAU/gds) | Hour | % Sol. | DS | Glucose | DP2 | DP3 | (>DP3) |
| Experiment #1 | 3 | 51.48 | 21.64 ± 0.12 | 64.49 ± 0.26 | 16.15 ± 0.22 | 1.26 ± 0.01 | 18.10 ± 0.05 |
| 0.15 added at | 24 | 82.79 | 31.85 ± 0.11 | 94.51 ± 0.04 | 2.33 ± 0.001 | 0.96 ± 0.01 | 2.21 ± 0.04 |
| t = 0 hr | 48 | 90.77 | 34.19 ± 0.10 | 95.88 ± 0.02 | 2.69 ± 0.01 | 0.52 ± 0.003 | 0.90 ± 0.01 |
| (control) | 72 | 94.06 | 35.14 ± 0.11 | 95.56 ± 0.02 | 3.34 ± 0.01 | 0.46 ± 0.002 | 0.65 ± 0.01 |
| | 96 | 95.76 | 35.62 ± 0.09 | 95.07 ± 0.07 | 3.91 ± 0.03 | 1.02 ± 0.03 | 0 |
| | 103 | 96.24 | 35.75 ± 0.09 | 94.95 ± 0.03 | 3.99 ± 0.02 | 1.06 ± 0.05 | 0 |
| Experiment #2 | 3 | 36.38 | 15.82 ± 0.02 | 1.88 ± 0.03 | 9.83 ± 0.01 | 12.05 ± 0.06 | 76.24 ± 0.02 |
| 0.15 added at | 24 | 81.48 | 31.08 ± 0.02 | 94.19 ± 0.13 | 2.30 ± 0.04 | 1.12 ± 0.03 | 2.40 ± 0.06 |
| t = 5 hr | 48 | 91.74 | 34.06 ± 0.06 | 95.84 ± 0.03 | 2.72 ± 0.05 | 0.53 ± 0.04 | 0.91 ± 0.04 |
| | 72 | 95.28 | 35.06 ± 0.13 | 95.55 ± 0.09 | 3.43 ± 0.10 | 1.02 ± 0.01 | 0 |
| | 96 | 97.48 | 35.67 ± 0.00 | 95.00 ± 0.25 | 4.00 ± 0.25 | 1.00 ± 0.001 | 0 |
| | 103 | 97.77 | 35.75 ± 0.14 | 94.70 ± 0.13 | 4.23 ± 0.17 | 1.07 ± 0.04 | 0 |
| Experiment #3 | 3 | 36.34 | 15.83 ± 0.04 | 1.88 ± 0.02 | 9.81 ± 0.07 | 12.06 ± 0.02 | 76.24 ± 0.04 |
| 0.15 added at | 24 | 76.89 | 29.76 ± 0.08 | 91.65 ± 0.17 | 2.89 ± 0.10 | 1.64 ± 0.01 | 3.82 ± 0.06 |
| t = 10 hr | 48 | 89.84 | 33.59 ± 0.18 | 95.71 ± 0.06 | 2.53 ± 0.02 | 0.63 ± 0.01 | 1.14 ± 0.03 |
| | 72 | 94.02 | 34.78 ± 0.05 | 95.81 ± 0.02 | 3.16 ± 0.01 | 0.43 ± 0.003 | 0.60 ± 0.01 |
| | 96 | 96.22 | 35.39 ± 0.24 | 95.37 ± 0.26 | 3.66 ± 0.21 | 0.97 ± 0.05 | 0 |
| | 103 | 96.89 | 35.58 ± 0.30 | 95.00 ± 0.03 | 3.96 ± 0.03 | 1.05 ± 0.004 | 0 |

### Example 11

### Effect of Staging of H-GA/An-GA Blend

In Example 11, the enzyme staging strategy, as described in Example 9 & 10, is applied for the H-GA/An-GA blend to investigate if enzyme staging is beneficial under these reaction conditions. A 35% DS corn starch slurry was prepared using dry bag starch from Roquette and tap water, adjusting the pH to 4.9. The slurry was divided over Schott Duran bottles, preparing all experiments in duplicate, and part of the enzymes were added. The glucoamylase blend contains H-GA at a concentration of 0.075 GAU/gds blended with An-GA (OPTIDEX® L-400) at a concentration of 0.09 GAU/gds. This glucoamylase blend was dosed with varying timing in three experiments: 1) 100% dose at 0 hrs, 2) 100% dose at 6 hrs, 3) 10% dose at 0 hrs and the remaining 90% dose at 6 hrs, as indicated in Table 11. The full SPEZYME® XTRA dosage of 10 AAU g/DS was added from the start of hydrolysis. After the alpha-amylase addition, the flasks were incubated at 60° C. Samples were taken at different time intervals during incubation for determining the percent solubilization and sugar composition.

Displayed in Table 11 are average values and standard deviation measures of duplicate incubations in one experiment. Bold numbers in Table 11 display the solubilization% values that are higher than for the reference experiment #1 without enzyme staging, and the underlined numbers are the DP1 maximum and DP2 minimum values of each experiment. Table 11 demonstrates that under the chosen conditions, enzyme staging had no large effect on the DP1 maximum and DP2 minimum values, while it did increase the solubilization values considerably (see results at 69 and 76 hrs for all experiments). As such, partial or complete delayed addition of the glucoamylase blend improves process results by combining increased solubilization values with optimal DP1 and DP2% values.

TABLE 11

| | Sacch. | | | % composition of the sugar | | | |
|---|---|---|---|---|---|---|---|
| | Time, | | Supernatant | | | | Hr. Sugar |
| Dosages added | Hour | % Sol. | DS | Glucose | DP2 | DP3 | (>DP3) |
| Experiment #1 | 5 | 57.44 ± 0.33 | 25.88 ± 0.12 | 72.25 ± 0.18 | 11.48 ± 0.07 | 1.89 ± 0.06 | 14.39 ± 0.17 |
| At 0 hrs: | 21.5 | 79.89 ± 0.33 | 33.48 ± 0.10 | 92.46 ± 0.02 | 2.54 ± 0.02 | 1.48 ± 0.03 | 3.52 ± 0.03 |
| H-GA = 0.075 GAU/gds | 29 | 83.37 ± 0.17 | 34.57 ± 0.05 | 93.38 ± 0.14 | 3.03 ± 0.03 | 1.11 ± 0.01 | 2.49 ± 0.16 |
| An-GA\= 0.09 GAU/gds | 45 | 88.66 ± 0.19 | 36.19 ± 0.06 | 94.33 ± 0.12 | 3.22 ± 0.12 | 0.83 ± 0.01 | 1.61 ± 0.01 |
| | 53 | 90.11 ± 0.44 | 36.63 ± 0.13 | 94.92 ± 0.11 | 3.27 ± 0.06 | 0.68 ± 0.03 | 1.12 ± 0.15 |
| | 69 | 91.54 ± 0.35 | 37.05 ± 0.10 | 94.95 ± 0.09 | 3.60 ± 0.02 | 0.55 ± 0.02 | 0.90 ± 0.09 |
| | 76 | 92.48 ± 0.07 | 37.33 ± 0.02 | 94.77 ± 0.04 | 3.84 ± 0.04 | 0.55 ± 0.02 | 0.85 ± 0.06 |
| Experiment #2 | 5 | 39.58 ± 0.31 | 18.90 ± 0.02 | 2.39 ± 0.01 | 10.62 ± 0.05 | 13.53 ± 0.05 | 73.46 ± 0.12 |
| At 6 hrs: | 21.5 | 76.55 ± 0.63 | 32.30 ± 0.02 | 90.81 ± 0.38 | 2.91 ± 0.15 | 1.86 ± 0.08 | 4.43 ± 0.15 |
| H-GA = 0.075 GAU/gds | 29 | 81.75 ± 0.44 | 33.95 ± 0.06 | 92.76 ± 0.20 | 2.95 ± 0.02 | 1.36 ± 0.06 | 2.94 ± 0.13 |
| An-GA = 0.09 GAU/gds | 45 | 88.16 ± 0.49 | 35.91 ± 0.36 | 94.30 ± 0.12 | 3.13 ± 0.02 | 0.88 ± 0.04 | 1.69 ± 0.10 |
| | 53 | 89.71 ± 0.08 | 36.38 ± 0.24 | 94.70 | 3.27 | 0.72 | 1.31 |
| | 69 | 92.04 ± 0.01 | 37.07 ± 0.22 | 94.79 ± 0.30 | 3.63 ± 0.20 | 0.60 ± 0.05 | 0.99 ± 0.05 |
| | 76 | 92.84 ± 0.07 | 37.31 ± 0.24 | 94.78 ± 0.03 | 3.76 ± 0.07 | 0.53 ± 0.06 | 0.92 ± 0.03 |
| Experiment #3 | 5 | 42.66 ± 0.19 | 20.21 ± 0.04 | 21.22 ± 0.29 | 14.43 ± 0.15 | 18.24 ± 0.02 | 46.12 ± 0.41 |
| At 0 hrs: | 21.5 | 76.58 ± 0.46 | 32.41 ± 0.09 | 91.38 ± 0.35 | 2.72 ± 0.08 | 1.77 ± 0.05 | 4.12 ± 0.23 |
| H-GA = 0.0075 GAU/gds | 29 | 81.53 ± 0.57 | 33.98 ± 0.11 | 92.89 ± 0.25 | 2.94 ± 0.03 | 1.32 ± 0.06 | 2.85 ± 0.15 |
| An-GA = 0.009 GAU/gds | 45 | 88.37 ± 0.65 | 36.08 ± 0.13 | 94.39 ± 0.27 | 3.13 ± 0.03 | 0.87 ± 0.07 | 1.62 ± 0.17 |
| At 6 hrs: | 53 | 89.95 ± 0.68 | 36.56 ± 0.14 | 94.67 ± 0.03 | 3.28 ± 0.08 | 0.74 ± 0.03 | 1.31 ± 0.08 |
| H-GA = 0.0675 GAU/g ds | 69 | 92.32 ± 0.47 | 37.26 ± 0.07 | 94.95 ± 0.13 | 3.60 ± 0.08 | 0.56 ± 0.06 | 0.89 ± 0.16 |
| An-GA = 0.081 GAU/g ds | 76 | 93.39 ± 0.11 | 37.58 ± 0.04 | 94.81 ± 0.02 | 3.81 ± 0.11 | 0.55 ± 0.03 | 0.83 ± 0.10 |

### Example 12

In the direct starch to glucose process, alpha-amylase is added together with the glucoamylase. Alpha-amylase and glucoamylase work synergistically on the starch granules, and alpha-amylase will produce substrates (oligosaccharides) for the glucoamylase to produce glucose. SPEZYME® XTRA (Bacillus stearothermophilus alpha-amylase) has been used so far. In this experiment, SAS3 was used as an alpha-amylase, replacing SPEZYME® XTRA. SAS3 is a DP4 producing alpha-amylase from Pseudomonas saccharophilu (Optimalt® 4G, Genencor-Danisco). By comparing the SAS3 and SPEZYME® XTRA experiment, we can see the effect of the changed oligosaccharide spectrum on the performance of the glucoamylase.

In this example, SPEZYME® XTRA (10 AAU/gds) alpha-amylase was replaced with SAS3 alpha-amylase (0.03 or 0.1 BMK/gds). BMK activity was determined using the Megazyme R-BAMR6 kit. One BMK unit equals 1000 Betamyl units and one Betamyl unit equals the release of 0.0351 mmole per 1 min. of p-nitrophenol.

The substrate was granular bag corn starch from Roquette, obtained via Barentz. A 35% DS corn starch slurry was prepared using dry bag starch and tap water. Slurry was incubated at 60° C. at pH 4.9, 0.075 GAU/gds H-GA and 0.09 GAU/gds An-GA, and the alpha-amylase. Samples were taken at different time intervals during incubation for determining the percent solubilization and sugar composition.

Displayed are average values and standard deviation. SPEZYME® XTRA (reference) is shown at the top of Table 12 and below that the two doses of SAS3 are displayed. Italic numbers display values that are lower than the reference values, bold numbers display values that are higher than the reference.

TABLE 12

| Alpha- | Sacch. | | | % composition of the sugar | | | |
|---|---|---|---|---|---|---|---|
| amylase type | Time, | | Supernatant | | | | Hr.Sugar |
| and dose | Hour | % Sol. | DS | Glucose | DP2 | DP3 | (>DP3) |
| SPEZYME ® | 5 | 57.4 ± 0.3 | 25.9 ± 0.1 | 72.25 ± 0.18 | 11.48 ± 0.07 | 1.89 ± 0.06 | 14.39 ± 0.17 |
| XTRA, 10 | 21.5 | 79.9 ± 0.3 | 33.5 ± 0.1 | 92.46 ± 0.02 | 2.54 ± 0.02 | 1.48 ± 0.03 | 3.52 ± 0.03 |
| AAU/gds | 29 | 83.4 ± 0.2 | 34.6 ± 0.1 | 93.38 ± 0.14 | 3.03 ± 0.03 | 1.11 ± 0.01 | 2.49 ± 0.16 |
| | 45 | 88.7 ± 0.2 | 36.2 ± 0.1 | 94.34 ± 0.12 | 3.22 ± 0.12 | 0.83 ± 0.01 | 1.61 ± 0.01 |
| | 53 | 90.1 ± 0.4 | 36.6 ± 0.1 | 94.92 ± 0.11 | 3.27 ± 0.06 | 0.69 ± 0.03 | 1.12 ± 0.15 |
| | 69 | 91.5 ± 0.3 | 37.1 ± 0.1 | 94.95 ± 0.09 | 3.60 ± 0.02 | 0.55 ± 0.02 | 0.90 ± 0.09 |
| | 76 | 92.5 ± 0.1 | 37.3 ± 0.0 | 94.77 ± 0.04 | 3.84 ± 0.04 | 0.55 ± 0.02 | 0.85 ± 0.06 |
| SAS3, 0.03 | 5 | 31.1 ± 1.4 | 15.2 ± 0.6 | 91.21 ± 1.52 | 2.68 ± 0.45 | 0.53 ± 0.16 | 5.59 ± 1.23 |
| BMK/gds | 21.5 | 47.4 ± 0.9 | 22.0 ± 0.3 | 95.88 ± 0.27 | 2.40 ± 0.07 | 0.19 ± 0.02 | 1.54 ± 0.18 |
| | 29 | 50.6 ± 1.0 | 23.2 ± 0.3 | 96.18 ± 0.31 | 2.47 ± 0.16 | 0.13 ± 0.03 | 1.23 ± 0.12 |
| | 45 | 56.8 ± 1.6 | 25.5 ± 0.5 | 96.26 ± 0.23 | 2.70 ± 0.07 | 0.14 ± 0.00 | 0.90 ± 0.16 |
| | 53 | 58.9 ± 1.4 | 26.2 ± 0.5 | 96.27 ± 0.05 | 2.82 ± 0.01 | 0.12 ± 0.03 | 0.79 ± 0.04 |
| | 69 | 61.9 ± 2.4 | 27.3 ± 0.8 | 95.82 ± 0.14 | 3.08 ± 0.05 | 0.21 ± 0.00 | 0.89 ± 0.08 |
| | 76 | 63.3 ± 2.5 | 27.8 ± 0.8 | 96.07 ± 0.23 | 3.15 ± 0.01 | 0.16 ± 0.00 | 0.61 ± 0.22 |
| SAS3, 0.1 | 5 | 33.2 ± 1.4 | 16.1 ± 0.7 | 89.66 ± 1.00 | 3.65 ± 0.46 | 0.58 ± 0.03 | 6.11 ± 0.52 |
| BMK/gds | 21.5 | 50.7 ± 1.1 | 23.1 ± 0.5 | 95.64 ± 0.56 | 2.72 ± 0.31 | 0.23 ± 0.02 | 1.41 ± 0.28 |
| | 29 | 54.1 ± 1.0 | 24.3 ± 0.5 | 95.76 ± 0.22 | 2.94 ± 0.08 | 0.18 ± 0.02 | 1.12 ± 0.16 |
| | 45 | 59.9 ± 1.1 | 26.4 ± 0.5 | 95.98 ± 0.26 | 3.06 ± 0.15 | 0.16 ± 0.02 | 0.80 ± 0.09 |
| | 53 | 61.8 ± 1.4 | 27.1 ± 0.6 | 95.97 ± 0.29 | 3.21 ± 0.09 | 0.16 ± 0.02 | 0.65 ± 0.18 |
| | 69 | 65.1 ± 1.3 | 28.2 ± 0.6 | 95.72 ± 0.05 | 3.44 ± 0.04 | 0.21 ± 0.02 | 0.63 ± 0.07 |
| | 76 | 66.3 ± 1.2 | 28.6 ± 0.5 | 95.50 ± 0.08 | 3.56 ± 0.05 | 0.26 ± 0.01 | 0.69 ± 0.02 |

SAS3 underperforms compared to SPEZYME® XTRA, as it shows much lower solubilization. Increased dose of SAS3 results in a slight increase in solubilization, but performance is still poor compared to SPEZYME® XTRA. DP1 levels with SAS3 are generally high, but in combination with the low solubilization the result is a low DP1 concentration (g/L).

### Example 13

### Effect of Glucoamylase Variants (Blends)

In the direct starch to glucose process, a blend of two glucoamylases (An-GA and H-GA) has been shown to give superior performance compared to either of the enzymes alone. The blend yields comparable or even higher solubilization as H-GA alone, whereas reversion reaction (DP2 levels) is considerably less, albeit not as low as An-GA alone. It has been found that a blend of 50:50 on activity basis is the optimum. In this example, blends were made by either replacing the H-GA with another glucoamylase, or replacing the An-GA with another glucoamylase.

All glucoamylase blends were dosed at the same total activity (0.328 GAU/gds), so 0.164 GAU/gds of each glucoamylase. A 35% DS corn starch slurry was prepared using dry bag starch and tap water. Slurry was incubated at 60° C. at pH 4.9, 10 AAU/gds SPEZYME® XTRA and the below mentioned types of glucoamylase. Samples were taken at different time intervals during incubation for determining the percent solubilization and sugar composition. Displayed are average values and standard deviations (duplicate incubations).

Table 13 contains data of the different glucoamylase blends. Displayed are average values and standard deviation. H-GA alone (reference) is shown at the top of Table 13 and below that the different blends of either H-GA with a varying second glucoamylase, or An-GA with another second glucoamylase (last series). Italic numbers display values that are lower than the reference values, bold numbers display values that are higher than the reference.

TABLE 13

| | Sacch. | | | % composition of the sugar | | | |
|---|---|---|---|---|---|---|---|
| Glucoamylase | Time | | Supernatant | | | | Hr. Sugar |
| type | Hour | % Sol. | DS | Glucose | DP2 | DP3 | (>DP3) |
| Humicola | 18 | 78.0 ± 1 | 32.4 ± 0.0 | 93.75 ± 0.0 | 2.58 ± 0.0 | 1.18 ± 0.0 | 2.49 ± 0.0 |
| | 25 | 82.5 ± 2 | 33.8 ± 0.0 | 95.00 ± 0.0 | 2.42 ± 0.0 | 0.93 ± 0.0 | 1.65 ± 0.0 |
| | 42.5 | 86.6 ± 1 | 35.1 ± 1.1 | 95.96 ± 0.0 | 2.64 ± 0.0 | 0.64 ± 0.0 | 0.75 ± 0.0 |
| | 49.5 | 88.2 ± 1 | 35.6 ± 1.0 | 96.15 ± 0.0 | 2.75 ± 0.0 | 0.55 ± 0.0 | 0.55 ± 0.0 |
| | 66.5 | 90.5 ± 0 | 36.2 ± 0.9 | 95.97 ± 0.0 | 3.22 ± 0.0 | 0.47 ± 0.0 | 0.34 ± 0.0 |
| | 73.5 | 91.4 ± 1 | 36.5 ± 1.0 | 95.89 ± 0.1 | 3.40 ± 0.0 | 0.43 ± 0.0 | 0.27 ± 0.0 |
| Humicola + | 18 | 76.8 ± 0 | 32.6 ± 0.1 | 93.24 ± 0.1 | 2.33 ± 0.0 | 1.32 ± 0.0 | 3.11 ± 0.0 |
| Aspergillus | 25 | 81.5 ± 0 | 34.1 ± 0.1 | 94.43 ± 0.1 | 2.21 ± 0.0 | 1.10 ± 0.0 | 2.26 ± 0.1 |
| niger | 42.5 | 87.3 ± 0 | 35.9 ± 0.2 | 95.87 ± 0.0 | 2.30 ± 0.0 | 0.75 ± 0.0 | 1.08 ± 0.0 |
| | 49.5 | 88.5 ± 0 | 36.2 ± 0.2 | 96.03 ± 0.0 | 2.45 ± 0.0 | 0.69 ± 0.0 | 0.83 ± 0.0 |
| | 66.5 | 90.8 ± 0 | 36.9 ± 0.2 | 96.09 ± 0.0 | 2.84 ± 0.0 | 0.53 ± 0.0 | 0.54 ± 0.0 |
| | 73.5 | 91.5 ± 0 | 37.1 ± 0.2 | 96.07 ± 0.0 | 2.97 ± 0.0 | 0.51 ± 0.0 | 0.45 ± 0.0 |
| Humicola + | 18 | 74.1 ± 0 | 31.4 ± 0.0 | 91.98 ± 0.0 | 2.90 ± 0.0 | 1.31 ± 0.0 | 3.80 ± 0.0 |
| Trichoderma | 25 | 78.5 ± 0 | 32.8 ± 0.0 | 93.72 ± 0.0 | 2.46 ± 0.0 | 1.14 ± 0.0 | 2.67 ± 0.0 |
| reesei | 42.5 | 84.1 ± 0 | 34.6 ± 0.2 | 95.49 ± 0.0 | 2.37 ± 0.0 | 0.78 ± 0.0 | 1.36 ± 0.0 |
| (Brew1) | 49.5 | 85.3 ± 0 | 34.9 ± 0.2 | 95.89 ± 0.1 | 2.42 ± 0.0 | 0.69 ± 0.0 | 1.00 ± 0.0 |
| | 66.5 | 87.4 ± 0 | 35.6 ± 0.2 | 96.15 ± 0.0 | 2.75 ± 0.0 | 0.51 ± 0.0 | 0.58 ± 0.0 |
| | 73.5 | 88.0 ± 0 | 35.8 ± 0.2 | 96.11 ± 0.0 | 2.89 ± 0.0 | 0.50 ± 0.0 | 0.51 ± 0.0 |
| Humicola + | 18 | 73.1 ± 0 | 31.5 ± 0.1 | 91.72 ± 0.3 | 3.04 ± 0.1 | 1.36 ± 0.0 | 3.89 ± 0.2 |
| Trichoderma | 25 | 77.3 ± 0 | 32.9 ± 0.2 | 93.50 ± 0.2 | 2.55 ± 0.0 | 1.18 ± 0.0 | 2.77 ± 0.1 |
| reesei | 42.5 | 82.7 ± 2 | 34.6 ± 0.6 | 95.55 ± 0.5 | 2.34 ± 0.1 | 0.78 ± 0.0 | 1.33 ± 0.2 |
| (Brew11) | 49.5 | 83.7 ± 2 | 34.9 ± 0.7 | 95.88 ± 0.4 | 2.37 ± 0.1 | 0.68 ± 0.1 | 1.07 ± 0.2 |
| | 66.5 | 85.9 ± 2 | 35.6 ± 0.6 | 96.17 ± 0.1 | 2.70 ± 0.0 | 0.51 ± 0.0 | 0.63 ± 0.0 |
| | 73.5 | 86.3 ± 2 | 35.7 ± 0.5 | 96.14 ± 0.1 | 2.82 ± 0.0 | 0.48 ± 0.1 | 0.55 ± 0.0 |
| Humicola + | 18 | 73.0 ± 0 | 31.3 ± 0.0 | 90.84 ± 0.0 | 3.57 ± 0.0 | 1.35 ± 0.0 | 4.24 ± 0.0 |
| Trichoderma | 25 | 77.1 ± 0 | 32.6 ± 0.0 | 92.83 ± 0.0 | 2.86 ± 0.0 | 1.25 ± 0.0 | 3.05 ± 0.0 |
| reesei | 42.5 | 81.6 ± 0 | 34.0 ± 0.1 | 95.35 ± 0.0 | 2.39 ± 0.0 | 0.83 ± 0.0 | 1.43 ± 0.0 |
| (CS4) | 49.5 | 82.7 ± 0 | 34.3 ± 0.1 | 95.76 ± 0.0 | 2.45 ± 0.0 | 0.69 ± 0.0 | 1.10 ± 0.0 |
| | 66.5 | 84.9 ± 0 | 35.1 ± 0.2 | 95.91 ± 0.0 | 2.76 ± 0.0 | 0.59 ± 0.0 | 0.74 ± 0.0 |
| | 73.5 | 85.6 ± 0 | 35.2 ± 0.2 | 95.95 ± 0.0 | 2.86 ± 0.0 | 0.54 ± 0.0 | 0.65 ± 0.0 |
| Spirizyme | 18 | 75.4 ± 0 | 32.0 ± 0.2 | 92.16 ± 0.2 | 2.21 ± 0.0 | 1.48 ± 0.0 | 4.15 ± 0.1 |
| Flex + | 25 | 78.5 ± 0 | 33.0 ± 0.3 | 93.82 ± 0.1 | 1.84 ± 0.0 | 1.35 ± 0.0 | 3.00 ± 0.1 |
| Aspergillus | 42.5 | 81.4 ± 1 | 34.0 ± 0.5 | 95.28 ± 0.1 | 2.13 ± 0.0 | 0.97 ± 0.0 | 1.62 ± 0.0 |
| niger | 49.5 | 81.8 ± 1 | 34.1 ± 0.5 | 95.52 ± 0.1 | 2.24 ± 0.0 | 0.84 ± 0.0 | 1.40 ± 0.0 |
| | 66.5 | 83.5 ± 1 | 34.6 ± 0.5 | 95.60 ± 0.0 | 2.66 ± 0.0 | 0.68 ± 0.0 | 1.06 ± 0.0 |
| | 73.5 | 83.8 ± 1 | 34.7 ± 0.5 | 95.69 ± 0.0 | 2.76 ± 0.0 | 0.61 ± 0.0 | 0.94 ± 0.0 |

Blending H-GA with An-GA results in increased solubilization, decreased DP2 and increased glucose levels towards the end of the process. Blending H-GA with another glucoamylase than An-GA still results in a reduced reversion reaction and increased glucose levels at the end of the process (albeit a lower glucose production rate), but also a loss in solubilization.

On the other hand, An-GA was blended with another (high reversion) glucoamylase than H-GA, Spirizyme® Flex in this case. This results in similar reversion reaction as the An-GA/H-GA blend, but slightly lower glucose levels and much lower solubilities.

What is claimed is:

1. A method of making a glucose syrup from refined granular starch slurry comprising;
contacting the refined granular starch slurry at a temperature at or below the initial starch gelatinization temperature with a dose of at least 8 AAU/gds of an alpha-amylase, and, a dose of 0.05 GAU/gds to no more than 0.3 GAU/gds of glucoamylase, and,
making a glucose syrup.

2. The method according to claim 1 wherein the glucose syrup comprises a DP1 of at least 90%.

3. The method according to claim 1 wherein at least 80% of the refined granular starch is solubilized.

4. The method according to claim 1 wherein the glucose syrup comprises a DP2 of less than 3%.

5. The method according to claim 1 wherein the refined granular starch slurry comprises an initial dry solids content (DS) of 31%-44% or 33-37%.

6. The method according to claim 1 wherein the glucoamylase comprises a mixture of glucoamylases, the mixture comprising a fast hydrolyzing glucoamylase and a low reversion glucoamylase.

7. The method according to claim 1 wherein the glucoamylase comprises a mixture of glucoamylases, wherein the mixture of glucoamylases comprises a fast hydrolyzing glucoamylase and a low reversion glucoamylase, wherein the fast hydrolyzing glucoamylase is Humicola glucoamylase and molecules 97% identical thereto, and the low reversion glucoamylase is A. Niger glucoamylase and molecules 97% identical thereto.

8. The method according to claim 1 further comprising treating with a pullulanase.

9. The method according to claim 1 wherein the pullulanase, if present, is Bacillus deramificans pullulanase and molecules 97% identical thereto.

10. The method according to claim 1 wherein a first dose of alpha-amylase is followed by a second dose of alpha-amylase, wherein the second dose occurs between 18 and 48 hours after the first dose.

11. The method according to claim 1 wherein a first dose of glucoamylase is followed by a second dose of glucoamylase, wherein the second dose occurs between 18 and 48 hours after the first dose.

12. The method according to claim 1 wherein a first dose of alpha-amylase is applied at a first temperature, and wherein the first temperature is elevated by 2° C.-8° C. after between 18 hours and 34 hours to a second temperature.

13. The method according to claim 1 wherein a first dose of glucoamylase is applied at a first temperature, and wherein the first temperature is elevated by 2° C.-8° C. after between 18 hours and 34 hours to a second temperature.

14. The method according to claim 1 wherein the alpha-amylase is selected from the group consisting of B. stearothermophilus, B. amyloliquefaciens and B. licheniformis, and molecules 97% identical thereto.

15. The method according to claim 1 wherein the alpha-amylase is B. stearothermophilus wild-type, or molecules 97% identical thereto.

16. The method according to claim 1 wherein the glucose syrup is made in less than 60 hours.

17. The method according to claim 1 wherein the refined starch is from corn, wheat, barley, rye, triticale, rice, oat, beans, banana, potato, sweet potato or tapioca.

18. The method according to claim 1 wherein the refined starch is from corn.

19. A composition comprising at least 8 AAU/gds of an alpha-amylase and 0.05 GAU/gds to no more than 0.3 GAU/gds of glucoamylase.

20. The composition according to claim 19 further comprising refined granular starch.

21-24. (canceled)
